(19) United States
(12) Patent Application Publication
(10) Pub. No.: US 2025/0288367 A1
(43) Pub. Date: Sep. 18, 2025

(54) SYSTEMS AND METHODS FOR SWITCHING CONTROL BETWEEN TOOLS DURING A MEDICAL PROCEDURE

(71) Applicant: INTUITIVE SURGICAL OPERATIONS, INC., Sunnyvale, CA (US)

(72) Inventors: Brandon D. Itkowitz, San Jose, CA (US); Sophia R. Hannaford, Palo Alto, CA (US); Paul W. Mohr, Mountain View, CA (US); Saleh Tabandeh, San Carlos, CA (US)

(21) Appl. No.: 18/858,666

(22) PCT Filed: Apr. 21, 2023
(86) PCT No.: PCT/US2023/019359

§ 371 Date: Oct. 21, 2024

Related U.S. Application Data
(60) Provisional application No. 63/333,974, filed on Apr. 22, 2022.

(51) Int. Cl. A61B 34/00; A61B 34/35; G16H 40/63

(52) U.S. Cl. CPC A61B 34/25; A61B 34/35; A61B 34/74; A61B 34/76; G16H 40/63

(57) ABSTRACT

A medical system comprises a display system, an input system including a first control device, and a control system. The control system includes a processing unit configured to display an image of a field of view of a surgical environment, determine a first keypoint on a first tool in the surgical environment, determine a selection region associated with the first keypoint of the first tool, determine a position of a cursor relative to the field of view that corresponds to a position of the first control device, and determine if the position of the cursor overlaps the selection region. The processing unit may also be configured to provide a directional cue if the position of the cursor overlaps the selection region to direct the cursor toward the first keypoint and engage the first control device with the first tool when the cursor reaches the first keypoint.

### CROSSED-REFERENCED APPLICATIONS

This application claims priority to and benefit of U.S. Provisional Application No. 63/333,974, filed Apr. 22, 2022 and entitled “Systems and Methods for Switching Control Between Tools During a Medical Procedure,” which is incorporated by reference herein in its entirety.

### FIELD

The present disclosure is directed to medical systems and methods for use in minimally invasive teleoperational medical procedures, and more particularly to systems and methods for switching assigned control between tools during a medical procedure.

### BACKGROUND

Minimally invasive medical techniques are intended to reduce the amount of extraneous tissue that is damaged during diagnostic or surgical procedures, thereby reducing patient recovery time, discomfort, and harmful side effects. Such minimally invasive techniques may be performed through natural orifices in a patient anatomy or through one or more surgical incisions. Through these natural orifices or incisions, clinicians may insert medical tools to reach a target tissue location. Minimally invasive medical tools include instruments such as therapeutic instruments, diagnostic instruments, and surgical instruments. Minimally invasive medical tools may also include imaging instruments such as endoscopic instruments that provide a user with a field of view within the patient anatomy.

Some minimally invasive medical tools may be robot-assisted including teleoperated, remotely operated, or otherwise computer-assisted. During a medical procedure, the clinician may be provided with a graphical user interface including an image of a three-dimensional field of view of the patient anatomy that may include one or more of the minimally invasive medical tools. For some minimally invasive, robot-assisted procedures, an operator may switch control assignment of an operator control device from one tool to another tool. Improved systems and methods are needed to present graphical user interface elements that enable an operator to efficiently and effectively change control assignments between tools.

### SUMMARY

The embodiments of the invention are best summarized by the claims that follow the description.

In one example embodiment, a medical system comprises a display system, an input system including a first control device, and a control system. The control system includes a processing unit configured to display an image of a field of view of a surgical environment, determine a first keypoint on a first tool in the surgical environment, determine a selection region associated with the first keypoint of the first tool, determine a position of a cursor relative to the field of view that corresponds to a position of the first control device, and determine if the position of the cursor overlaps the selection region. The processing unit may also be configured to provide a directional cue if the position of the cursor overlaps the selection region to direct the cursor toward the first keypoint and engage the first control device with the first tool when the cursor reaches the first keypoint.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 illustrates an operator of a medical system in an operator environment.

FIGS. 1A-1D illustrate a graphical user interface including an image of an endoscopic field of view with a cursor corresponding to a controller of an input system, according to some examples

FIGS. 2A-2D illustrate a graphical user interface including an image of a field of view provided by an imaging instrument while swapping control of tools in the field of view, according to some examples.

FIG. 3 is a control device haptic force profile according to some examples.

FIGS. 4A-4D illustrate various examples of an adaptive selection region based on the proximity of tools in the field of view, according to some examples.

FIGS. 5A-5B illustrate a graphical user interface including an image of a field of view while swapping control to a tool outside the field of view, according to some examples.

FIGS. 6A-6D illustrate a graphical user interface including an image of a field of view while swapping control to a virtual tool, according to some examples.

FIGS. 7A-7B illustrate a graphical user interface including an image of a field of view while a control device is initially in an unassigned state, according to some examples.

FIGS. 8A-8C illustrate a graphical user interface including an image of a field of view while swapping control of closely positioned tools, according to some examples.

FIGS. 9A-9E illustrate a graphical user interface including an image of a field of view while swapping control of multiple tools in the field of view, according to some examples.

FIG. 10A-10B illustrate a graphical user interface including an image of a field of view during an interrupted control swap, according to some examples.

FIG. 11A is a flow chart of a method for reassigning control of a tool, according to some examples.

FIG. 11B is a flow chart of a method for reassigning control of a tool, according to some examples.

FIG. 12 illustrates a schematic view of a medical system, according to some examples.

FIG. 13 is a perspective view of a manipulator assembly of the medical system of FIG. 12, according to some examples.

FIG. 14 is a front elevation view of an operator's console in a robot-assisted medical system, according to some examples.

FIG. 15 is a perspective view of an exemplary gimbaled device of the operator's console of FIG. 14 to control one or more arms and/or tools coupled of the robot-assisted medical system, according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In robot-assisted medical procedures, endoscopic images of the surgical environment may provide a clinician with a field of view of the patient anatomy and any medical tools located in the patient anatomy. During a medical procedure an operator may switch control assignment of a control device from one selected tool to another selected tool. A tool may be associated with a single hand control device at a time. To simplify, expedite, and/or improve operator confidence in the assignment swap, spatially co-located graphical indicators and haptic cues may be provided to guide selection of the tools.

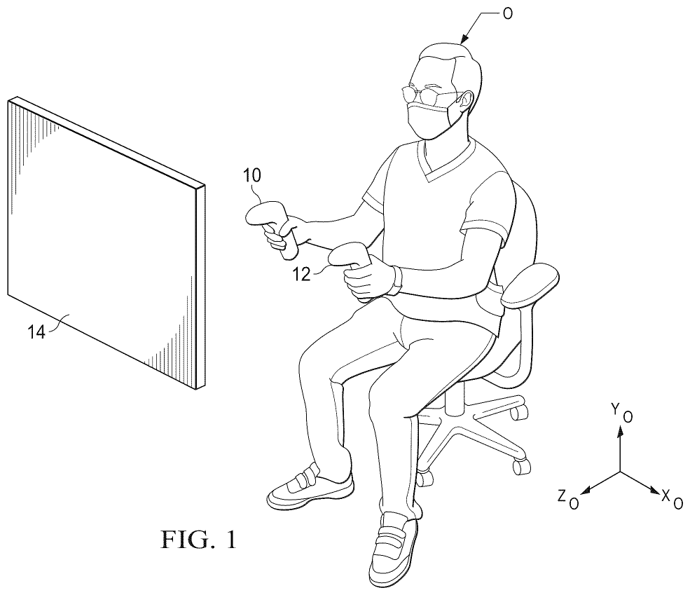

FIG. 1 illustrates a clinician or operator O in an operator environment, having an operator frame of reference X_{O}, Y_{O}, Z_{O}. The operator O may exercise control of a right-hand control device 10 and a left-hand control device 12 to control tools visible on a display system 14. In some examples the control devices 10, 12 may be part of a robot-assisted medical system such as medical system 310 described in greater detail below. The control device(s) may include one or more of any number of a variety of input devices, such as hand grips, joysticks, trackballs, data gloves, trigger-guns, hand-operated controllers, touch screens, body motion or presence sensors, and other types of input devices.

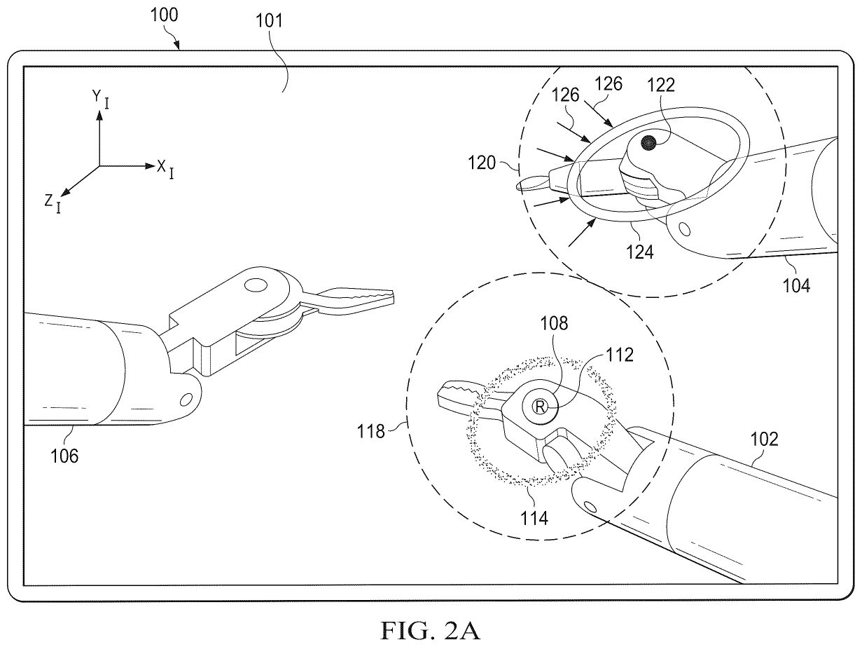

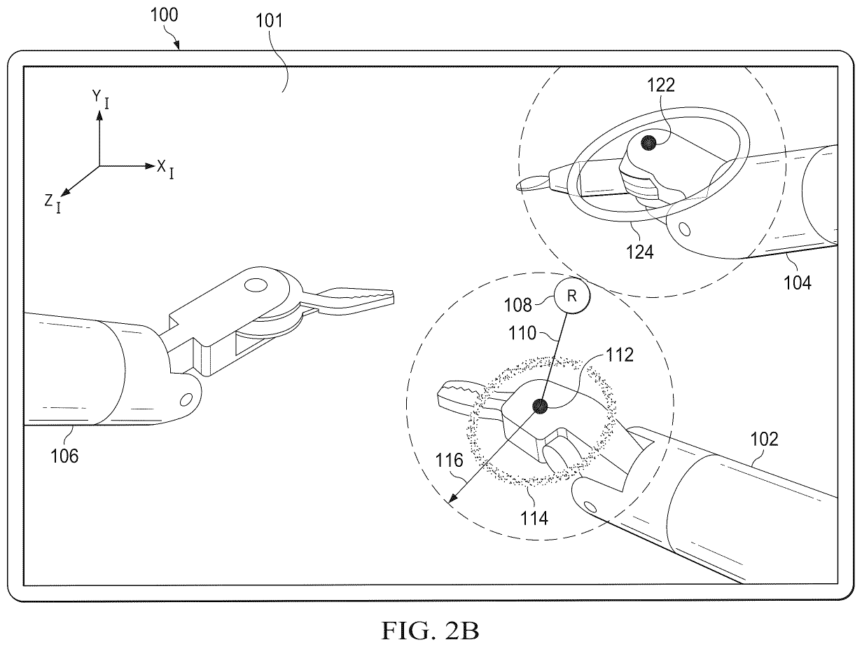

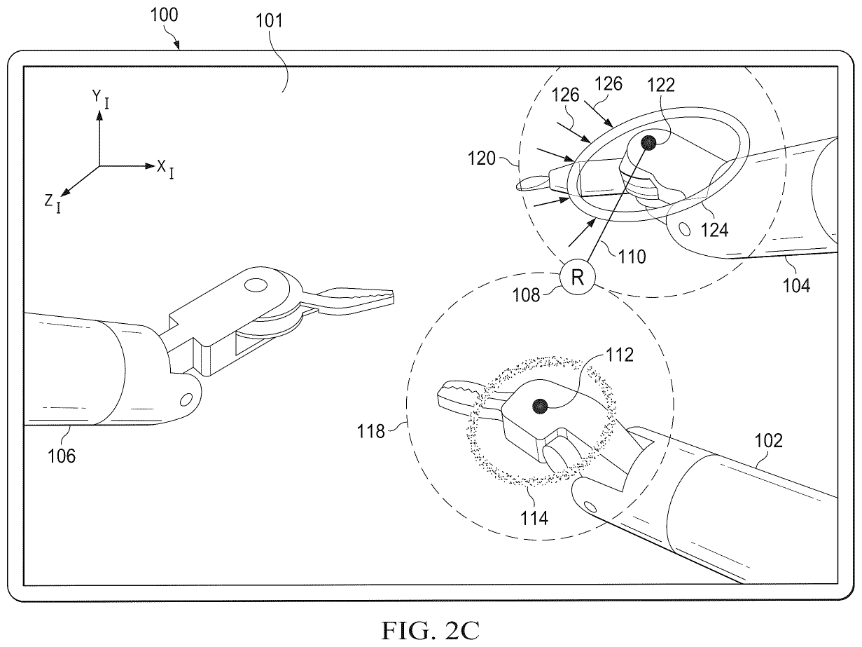

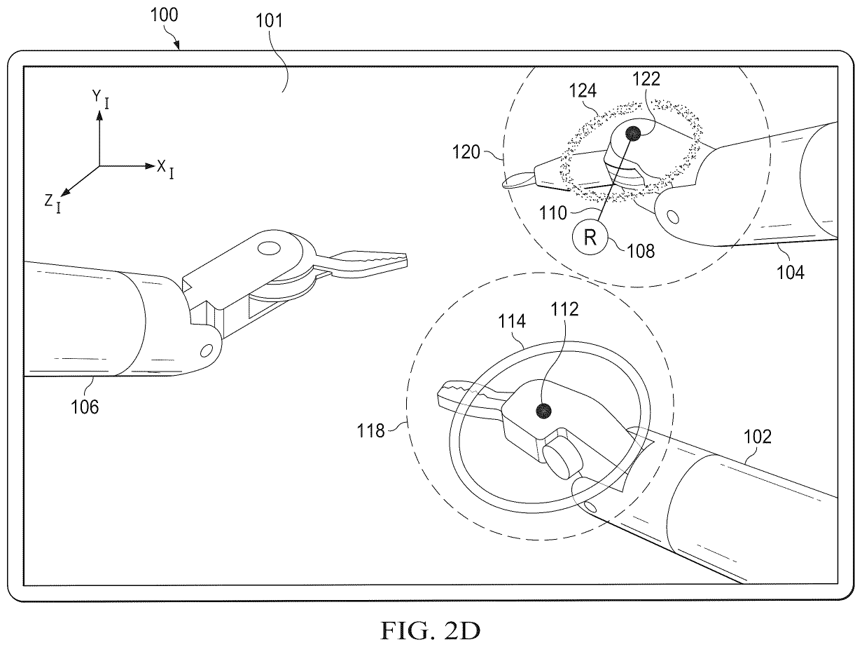

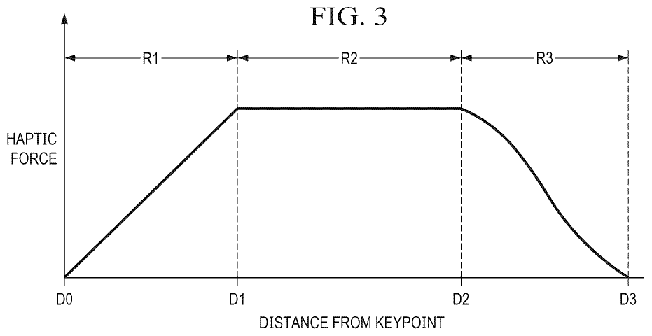

FIGS. 2A, 2B, 2C, and 2D together illustrate the reassignment of a control device between two tools visible in an image of a field of view displayed on a display system (e.g., display system 14). FIG. 2A illustrates graphical user interface 100 including an image 101 of a field of view provided by an imaging instrument or component (e.g., the endoscopic imaging system 315 described below) within an anatomic environment in a patient anatomy. In this example, the image 101 may be a three-dimensional, stereoscopic image, but in other examples, the image may be a two-dimensional image. The image 101 of the field of view may have an image frame of reference X_{I}, Y_{I}, Z_{I} based, for example, on the distal end of the endoscopic imaging system. The operator frame of reference X_{O}, Y_{O}, Z_{O} may be registered to the image frame of reference. This alignment of the reference frames allows hand motions in each of the cardinal directions to correspond to movement within the image. In this example, the field of view includes a tool 102, a tool 104, and a tool 106. Control of the tool 102 may initially be assigned to one or more at an operator input system of a robot-assisted medical system. For example, the right-hand control device 10 may control the tool 102 and the left-hand control device 12 may control the tool 106. When the robot-assisted medical system is in an instrument control mode or instrument “following” mode, the control devices 10, 12 may be movable in six degrees of freedom to provide corresponding movement to the tools in the anatomic environment.

To change the operator control device used to control a tool, the robot-assisted medical system may exit the instrument control mode and enter an instrument swap mode in which movement of the control device 10 no longer controls motion of the tool 102. In the instrument swap mode, spatially co-located graphic indicators and haptic cues may assist the operator in reassigning the control device 10 from controlling tool 102 to controlling tool 104. Rather than requiring movement of the control device 10 in six degrees of freedom to effect the swap, in the swap mode, movement of the control device 10 in two translational degrees of freedom (e.g., X,Y) may be recognized and transformed to control two-dimensional motion of a selection cursor 108. In the example of FIG. 2A, the cursor 108 is initially overlaps or is positioned within or in the vicinity of an adaptive selection region 118 centered on a keypoint 112. The adaptive selection region may include the keypoint 112 and may have an irregular or regular shaped boundary. In some examples, the adaptive selection region 118 may be defined by the sweep of an adaptive selection radius 116 extending from the keypoint 112. The adaptive selection region may be mapped to a normalized device coordinate (“NDC”) space of the single eyepiece of a stereoscopic imaging system. The cursor 108 may indicate to the operator which control device initially controls the tool. For example as shown in FIG. 2A, a cursor 108 may include a circular badge with an identification symbol such as the letter “R” indicating control of the tool 102 by the right-hand control device 10. In some examples, a circular badge with the letter “L” may indicate control of a tool by a left-hand control device 12. In some examples, the color of the cursor may indicate the identity of the current operator commanding the tool, in the case of multiple operators.

The cursor 108 may be rendered as a three-dimensional element that may initially appear to hover at a position in front of the tool 102. The position of the cursor relative to the field of view may be determined by mapping the cursor to a normalized device coordinate (“NDC”) space of a single eyepiece of a stereoscopic imaging system. The position of the cursor may be a two-dimensional perspective-projected position and may correspond to a two-dimensional perspective projection of the position of the control device 10 or a portion of the control device 10 (e.g., a distal portion). The two-dimensional position of the cursor 108 may be based on an affine transformation of the position of the control device 10. The affine transformation provides a scale and translation transformation between the control device reference frame (X_{O}, Y_{O}, Z_{O}) and the image frame of reference (X_{I}, Y_{I}, Z_{I}). The scale component of the transformation may be adaptively determined to optimize economy of motion and precision of selection for the operator. The translation component of the transformation may achieve an initial offset position for the cursor so it appears overtop of the currently selected too, in the image frame of reference.

As the control device 10 is moved in the operator environment, corresponding movement of the cursor 108 is shown on the graphical user interface 100. The movement of cursor 108 may occur in three dimensions but the tool selection process may rely on a two dimensional projected position to determine which tool is closest to the cursor. The three dimensional cursor presentation may allow the operator to maintain a comfortable stereoscopic fusion while the two dimensional selection criteria may make selection simpler and less sensitive to hand motions in the third dimension (e.g., depth). As shown in FIG. 2B, as the cursor 108 moves away from the tool 102, a graphical indicator line 110 or graphic zipline may extend between the cursor 108 and a keypoint 112 on the tool 102. The indicator line 110 may shorten as the cursor 108 approaches the keypoint 112 and may lengthen as the cursor 108 moves away from the keypoint 112. When multiple potential tools are candidates for selection on the graphical user interface, the indicator line 110 may serve as a visual preview of the closest target keypoint to the cursor 108.

A selection graphic 114, which in this example may be a selection hoop, identifies the selected tool 102. The selection hoop 114 may be rendered in three-dimensional appearance with the hoop centered on the keypoint 112 and extending generally normal to the surface of the tool 102 at the keypoint 112. The color and opacity of the selection hoop may change as the cursor approaches or withdraws from a candidate tool, providing an indication of the status of the instrument selection. For example, a translucent white selection hoop may indicate that the instrument 102 is not selected for control by the control device 10. An opaque hoop rendered in a selection color (e.g. colored cyan) may indicate that the instrument 102 has been selected for control by the control device 10. The translucency and color of the selection hoop may gradually change as the cursor 108 approaches and withdraws from various tools. In some examples, the color of the selection hoop may indicate the identity of the current operator commanding the tool, in the case of multiple operators. In other examples, other selection graphics including other shapes, shading, highlight, dynamic markers, or other graphics may be used as the selection graphic. The cursor 108, the indicator line 110, and/or the selection hoop 114 may allow a viewer to immediately understand which control device 10 is controlling the tool 102 while the robot-assisted medical system is in the instrument control mode.

As shown in FIG. 2C, as the control device 10 moves the cursor 108 toward the tool 104, the cursor 108 exits the adaptive selection region 118 and enters a selection region 120 centered on a keypoint 122 of the tool 104. As the cursor exits the adaptive selection region 118, the indicator line 110 becomes detached from the keypoint 112. The change in the termination of the indicator line from keypoint 112 to the keypoint 122 provides an indication that the control device 10 is changing from control of tool 102 to control of tool 104. As the cursor 108 moves toward and enters the adaptive selection region 120 (e.g., in view-apparent projected display coordinates), the indicator line 110 extends between the cursor 108 and the keypoint 122, the indicator line shrinking in size as the cursor nears the keypoint and lengthening as the cursor moves away from the keypoint. The indicator line 110 may snap to the closest adaptive selection region.

Movement of the cursor 108 and the control device 10 in the direction of the keypoint 122 may reinforce spatial relationships. The movement mapping of the control device 10 in the operator frame of reference to the cursor in the image frame of reference may include adaptive motion scaling. The scaling may ensure that the selectable keypoints are within the range of motion of the control device 10. A motion scaling factor may be limited to avoid excessive sensitivity and ensure that any motions are intentional. The motion scaling factor may also be limited to avoid excessive looseness and ensure that motion is within the workspace of the control device 10. In some examples, motion in the X-Y direction may have a different scale than the motion in the Z-direction since the distance between closest and farthest selection targets in the depth direction may be much greater than lateral separation.

As shown in FIG. 2D, with the cursor 108 overlaps or is within or in the vicinity of the selection region 120 for tool 104, a selection hoop 124 for the tool 104 changes from translucent to opaque, indicating that the right-hand control device 10 is being selected to control the tool 104. When the cursor 108 reaches the keypoint 122 or within a threshold distance thereof, the selection is complete and the control device 10 has control of the tool 104. Display of the cursor 108 may be suppressed (e.g., the cursor 108 may not be shown) after the selection and transfer are complete.

Each selection region 118, 120 may bound a haptic gravity well that haptically attracts the control device 10 and the cursor toward the keypoint. For example, the selection region 120 may bound a gravity well 126. When the cursor 108 enters the selection region 120 the gravity well 126 exerts a force on the control device 10 that attracts the control device and cursor toward the keypoint 122. More specifically, the gravity well 126 attracts the control device 10 toward a position in the operator frame of reference that corresponds to the position of the keypoint 122 in the image frame of reference. The haptic forces provided to the control device 10 while the cursor is in the selection region 120 may vary with the position of the cursor, dynamically determining stiffness and the damping properties. A haptic force profile may be applied based on the distance of the cursor 108 from the keypoint or based on the speed of motion of the control device 10 and cursor 108. For example and with reference to the haptic force profile of FIG. 3, the haptic force on the control device 10 may increase as the cursor 108 moves from distance D3 toward distance D2. Between a distance D1 and D2 from the keypoint 122, a constant force and/or damping may be applied to the control device 10. Between the distance D1 and the keypoint (D0), the haptic force may be gradually reduced. The haptic force profile may be determined or tuned so that a gentle pull or push force is applied to the control device 10, while avoiding forces that would pull the control device 10 out of the operator's hand. In some examples, the haptic force may terminate at a threshold distance from the keypoint, within or overlapping the adaptive selection region but before the cursor reaches the keypoint. The haptic force may include translational forces that urge translational motion of the control device 10 and may also include forces to modify the orientation of the control device or the finger grip to match the orientation and grip (e.g., the jaw closure angle) of the tool to which the cursor is being attracted.

In some examples, the region of the gravity well may extend a threshold distance from the distal boundary of the selection region. Generally, a graphical indication of the adaptive selection regions may be suppressed (e.g., not displayed) such that the selection region is only detectable by the presence of the indicator lines and the haptic attractive force of the gravity wells. In other examples, a boundary line, shading, or other graphical treatment may be displayed on the graphical user interface 100 to indicate the location of the selection regions.

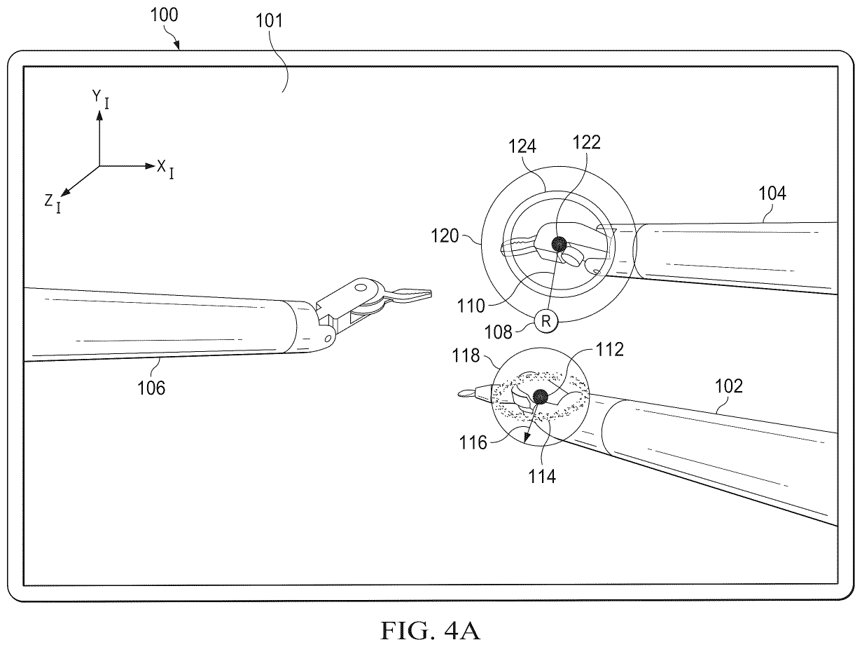

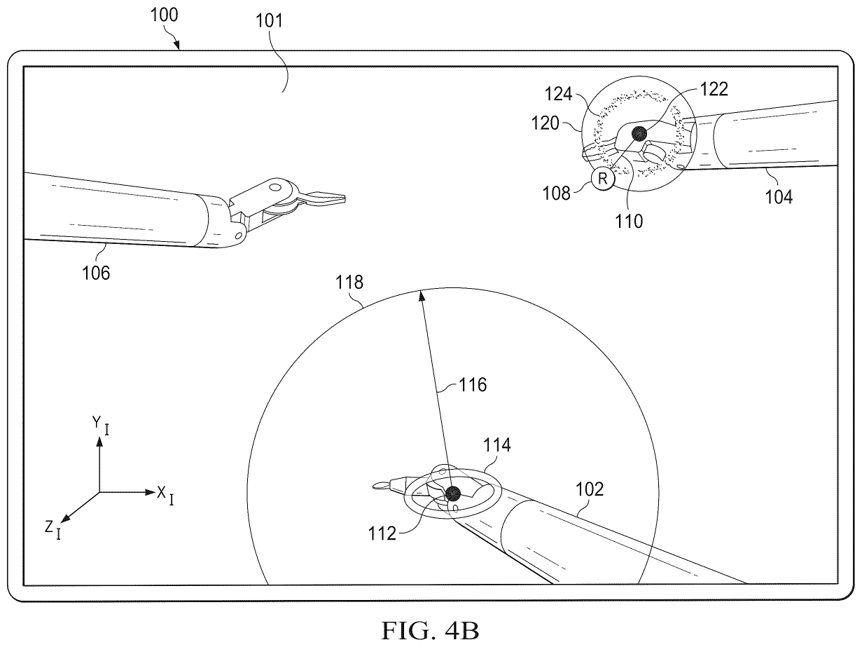

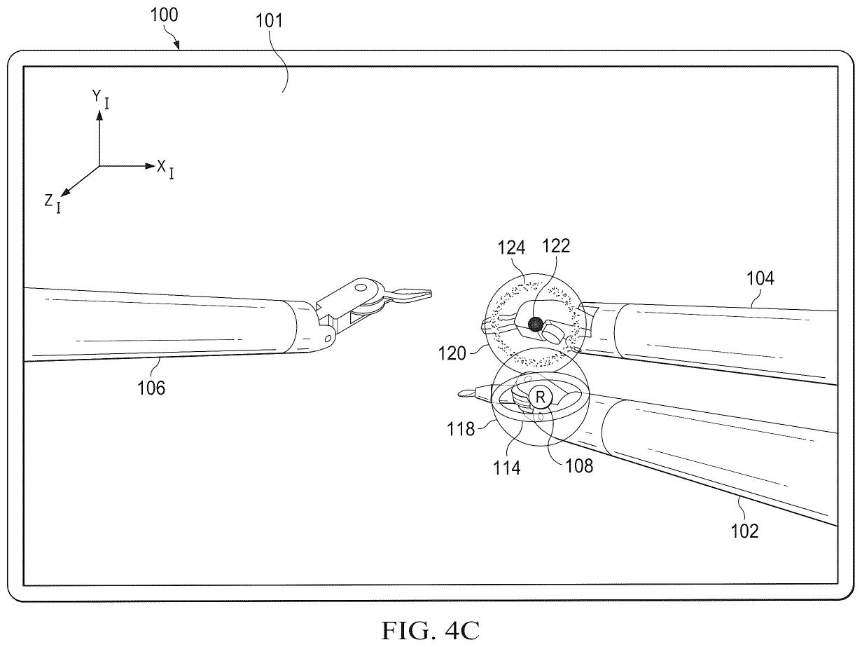

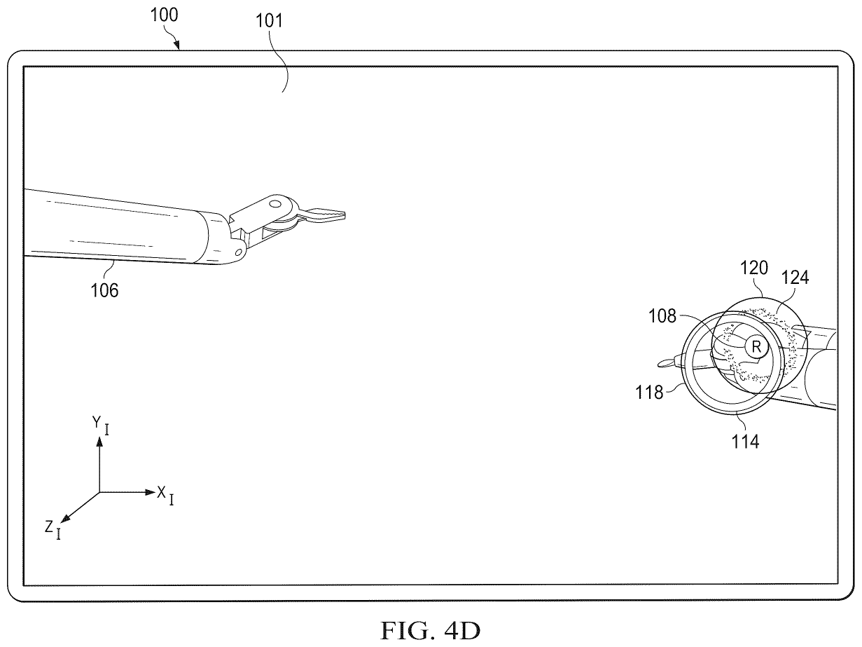

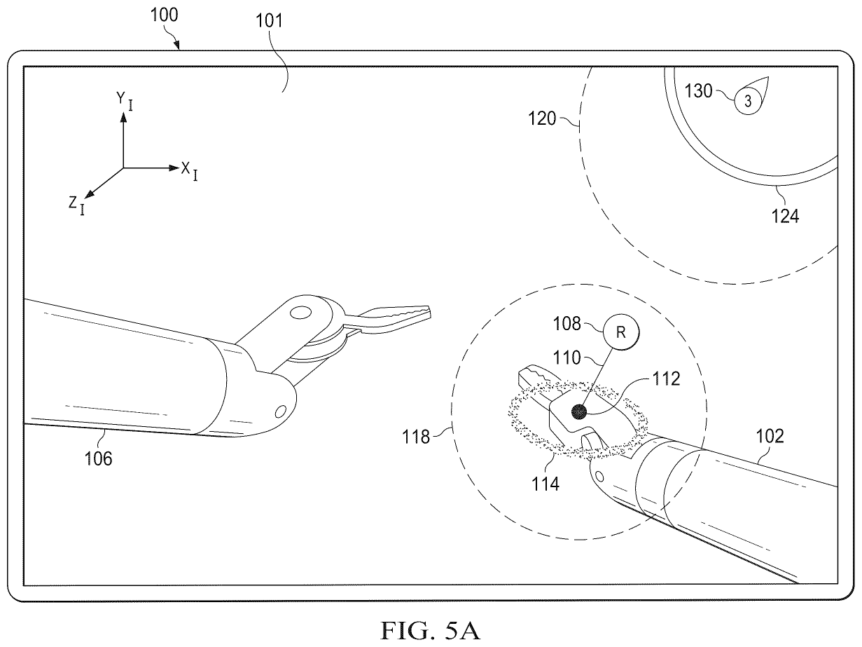

FIGS. 4A-4D illustrate the adaptive nature of the selection regions. As shown in FIGS. 4A and 4B, the size of the adaptive selection region 118 and its radius 116 may vary based on the proximity of other candidate tools. Additionally or alternatively, the radius 116 may be sized to avoid intersecting with possible cursor trajectories between instruments. In FIG. 4B, with the tool 102 moved farther away from tool 104, as compared to FIG. 4A, the adaptive section region 118 and the radius 116 becomes larger as compared to FIG. 4A where the tools are more closely spaced. The larger adaptive selection region 118 corresponds to a larger gravity well for the tool 102 in FIG. 4B. Thus, when control is swapped from tool 104 to tool 102, the larger gravity will for tool 102 will begin attracting the control device 10 and cursor 108 toward the keypoint 112 sooner on the trajectory from tool 104 to 102 (i.e., at a greater distance from the keypoint 112). The larger selection region may allow the selection experience to become more gestural and therefore faster, easier, and more accurate. As previously described, in some examples, the outline of the adaptive selection regions 118, 120 may be suppressed on the display. The adaptive selection regions 118, 120 may have a default radius size, a minimum radius size, and a maximum radius size.

As shown in FIG. 4C and FIG. 4D, an appearance of the boundary of the adaptive selection regions 118, 120 may be altered as the keypoints become close to each other and the adaptive selection regions begin to overlap. If displayed, a changed appearance of the selection region boundary may include a change in color, texture, opacity, or another visually discernable change to indicate a warning about the close proximity of the selection regions. In some examples, the operator control device may also provide additional viscous resistance to improve precision of selection and to reduce overshoot. In FIG. 4C, the boundaries of the selection regions 118, 120 overlap but the keypoints 112, 122 remain in distinctly different selection regions. The selection region boundaries may be depicted in the color orange to indicate a warning to the viewer that although the cursor 108 and control device 10 are assigned to tool 102, they are in potentially confusing close proximity to tool 104. In some examples, overlap or close proximity may be tracked as a diagnostic aid, without presentation (e.g., by color coding) to the viewer. For example, if an overlap is detected, the operator may be directed, cued, or otherwise influenced to move the cursor outside of the first selection region before a second selection region may be selected. This may ensure a minimum hand travel distance before a selection change occurs. The selection hoop 124 is rendered in the selection color but is translucent indicating that the tool 104 was assigned to the control device 10 but the cursor 108 has moved out of the selection region 120 toward another tool.

The process for determining selection regions may first try to identify keypoint or locations on each tool that will eliminate overlap between selection regions. In some examples, however, there may be no alternative non-overlapping regions. For example, in FIG. 4D the tool 104 is positioned behind the tool 102 and the boundaries of the selection regions 118, 120 overlap with the keypoints 112, 122 positioned inside both selection regions 118, 120. Thus, a viewer would have difficulty or be unable to determine, based on visual appearance of the cursor alone, the tool to which the cursor 108 is assigned. The selection region boundaries may be depicted in the color red to indicate a heightened warning to the viewer that the cursor 108 may not unambiguously identify the tool to which the right-hand control device 10 is assigned. The warning color may also indicate that motion in a Z-direction (i.e., depth) is now activated, allowing the operator an additional degree of translation motion to for selection of the tool. In FIG. 4D, the opaque selection hoop 124 for tool 104 may provide further indication that the control device 10 is assigned to tool 104. In an alternative example, other visible portions of the tools that are not overlapping may be used as selection targets.

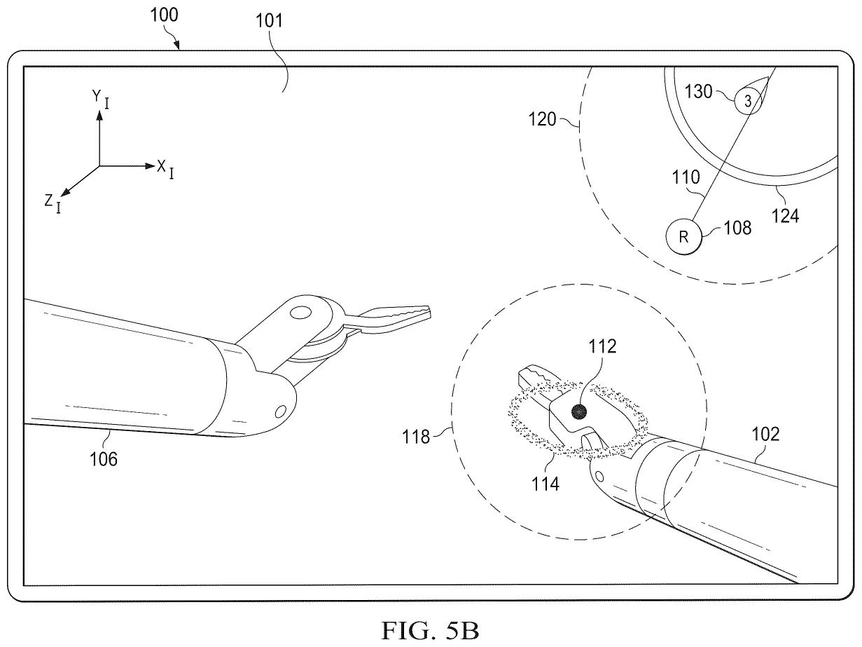

As shown in FIGS. 5A and 5B, the control device 10 may be assigned to off-screen tools. In FIG. 5A, the control device 10 may be assigned to the tool 102 as indicated by the opaque, colored selection hoop 114, and the cursor 108 may be tethered to the keypoint 112 by the indicator line 110. To reassign the control device 10 to tool 104, which is outside of the image 101 of the field of view but indicated with a marker 130, the cursor 108 is moved toward the selection region 120 which may be centered at the marker 130 or, alternatively, at the off-screen keypoint 122. The selection region may be constrained to appear at the edge of the image 101 of the field of view closest to the tool. In some examples, the marker 130 may include the number of the manipulator arm to which the tool is coupled.

As shown in FIG. 5B, with the cursor 108 moved inside the selection region 120, the indicator line 110 extends from the cursor 108 to marker 130. The gravity well within the selection region 120 may provide an attractive force that urges the control device 10 to move the cursor 108 toward the marker 130. As the cursor 108 nears the marker 130, the indicator line 110 shrinks until the cursor is located at the marker 130. As the cursor 108 moves through the selection region 120, the selection hoop may transition from translucent to opaque and may change from white to the selection color.

In some examples, the imaging system may be repositioned to view the tool 104. In some examples, the adaptive selection region (and adaptive selection radius) may be reduced in size as compared the adaptive selection regions for tools in the field of view to require a more intentional selection motion for off-screen tools.

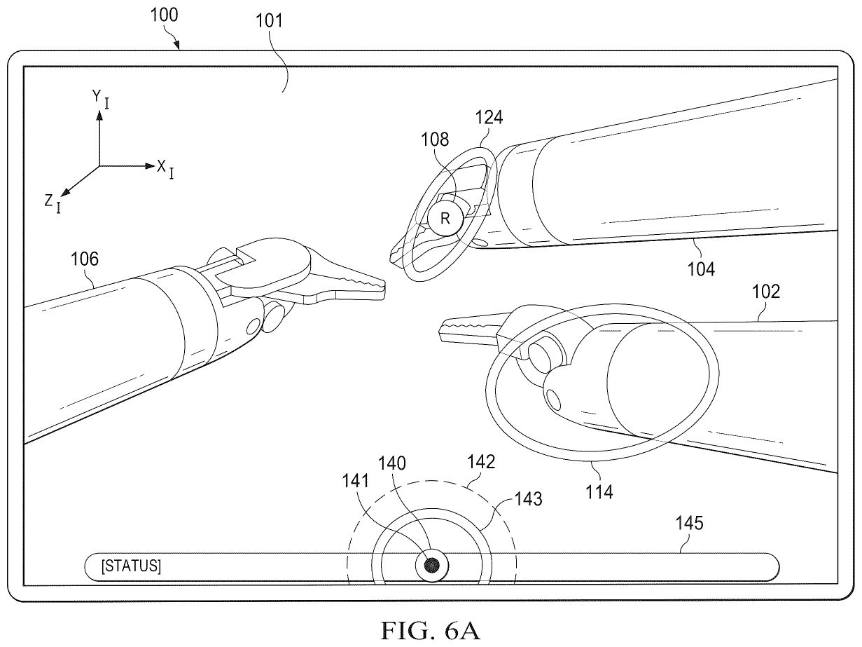

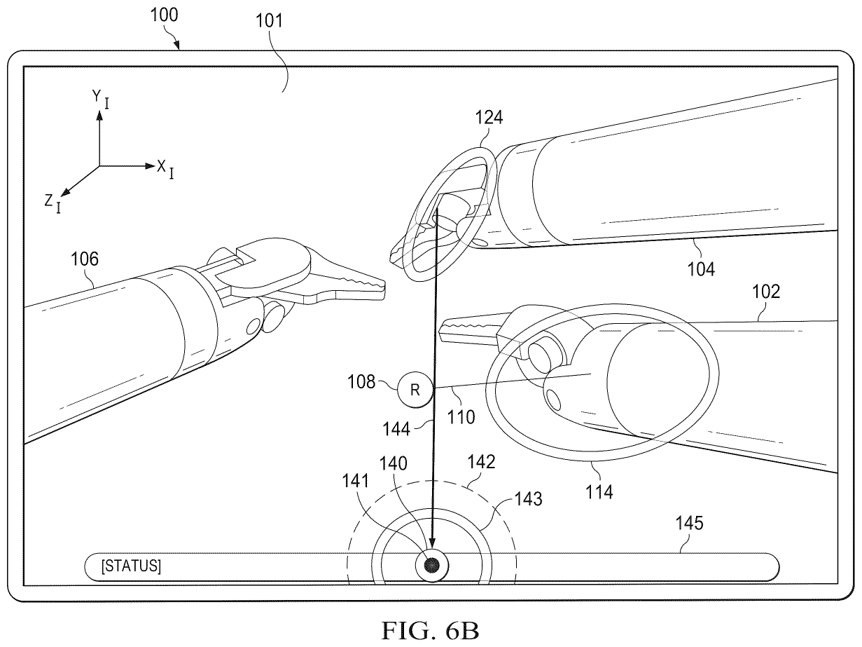

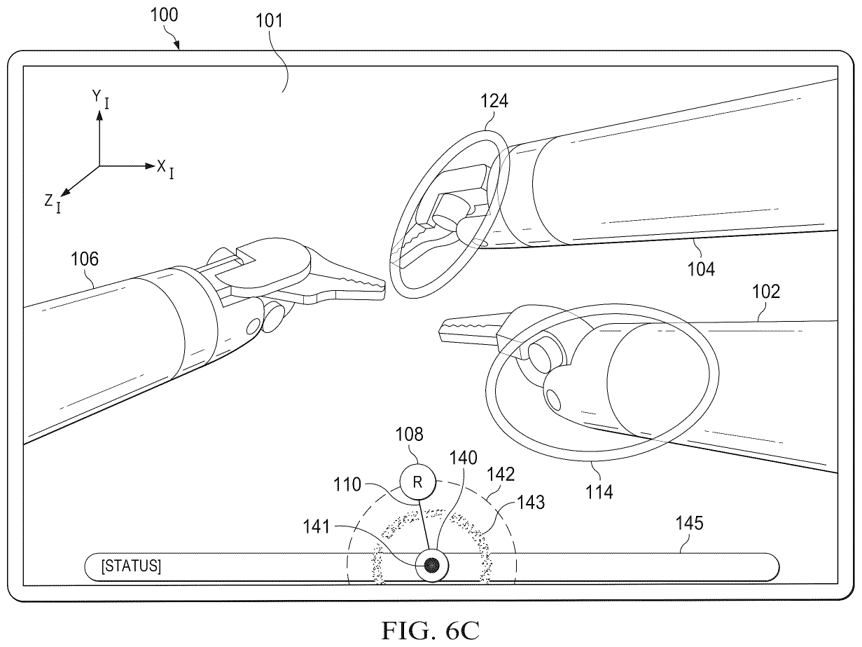

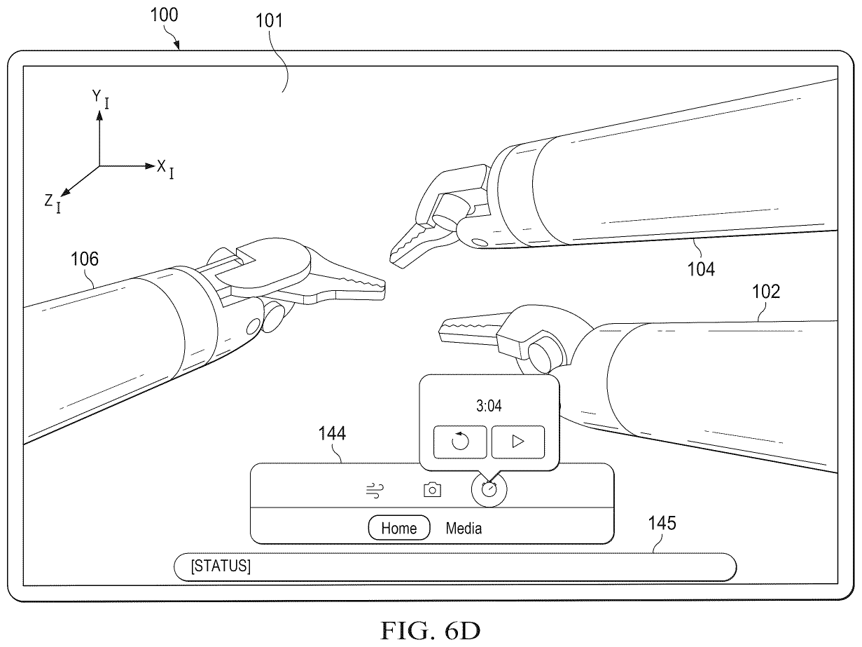

As shown in FIGS. 6A-6B, the cursor may temporarily snap to intermediate tools encountered on the trajectory to a destination tool. In this example, the destination tool may be a virtual tool such as a menu. In FIG. 6A, the cursor 108 is snapped to the tool 104 and the selection hoop is opaque and rendered in the selection color, indicating the control device 10 has control of tool 104. The operator may choose to transfer control from the tool 104 to a virtual tool 140, such as a pop-up on-screen menu with a controllable cursor for navigating the on-screen menu. The virtual tool 140 may have a selection region 142 and a selection hoop 143 centered on a keypoint 141. In the swap mode, as the cursor 108 moves from the keypoint 122 for tool 104 along a trajectory or flight path 144 toward the keypoint 141, the cursor 108 may briefly enter the selection region 118 for intermediate tool 102, causing the indicator line 110 to snap to (i.e., have a transitory termination at) the keypoint 112 of the tool 102 as shown in FIG. 6B. A haptic attractive force within the selection region of the tool 102 may attract the cursor 108 toward the tool 102. In some examples, the control system may identify possible trajectories between the cursor and multiple potential target keypoints. In some examples, the control system may optimize the choice of target keypoints and their respective adaptive selection radii to reduce selection region overlap and overlap with potential trajectories. If some overlap remains in the placement and sizing of the selection regions, the operator may be able to resist the haptic attraction forces to the intermediate tool keypoint in order to leave the intermediate selection region and move toward the desired tool.

As the control device 10 continues to move the cursor 108 toward the keypoint 141 for the virtual tool, 140, the indicator line 110 snaps to the keypoint 141 when the cursor 108 enters a selection region 142 for the keypoint 141 as shown in FIG. 6C. The indicator line 110 shrinks as the cursor approaches the keypoint 141. A gravity well within the selection region 142 may provide a haptic force on the control device 10 that attracts the cursor 108 toward the keypoint 141. The physical interaction of moving the cursor (e.g., a proxy for the hand) from one tool to another may convincingly aid the operator in disassociating and relaxing grip control of the former tool such that grip can be used as a control input in a menu system. In some examples, the location of the menu selection region may be adaptable to avoid overlapping the selection region of imaged tools. In various examples, the menu selection region may appear anywhere along the length of a displayed status bar 145. In some examples, a portion or the entire status bar 145 may be a selection region for the menu system with a haptic barrier that must be penetrated to access the menu. Such examples may provide an economy of motion since the user may be attempting to reach for a particular part of the menu system. The haptic barrier may also help to make entry to the menu system distinct from the selection of a tool.

In FIG. 6D, with the control device 10 assigned to the virtual tool 140, the virtual tool 140 may be manipulated to access features of a menu. The menu may, for example, provide access to controls and settings for an imaging system, an illumination system, an insufflation system, or any other system that may support the medical procedure. In some examples, each manipulator arm and/or instrument or tool may include a menu with menu options and settings (e.g., electrocautery settings, force feedback settings) particular to the arm and/or instrument. When swapping from the virtual menu tool to an imaged tool, haptic forces may be provided to the control device 10 to guide the control device to the original tool pose.

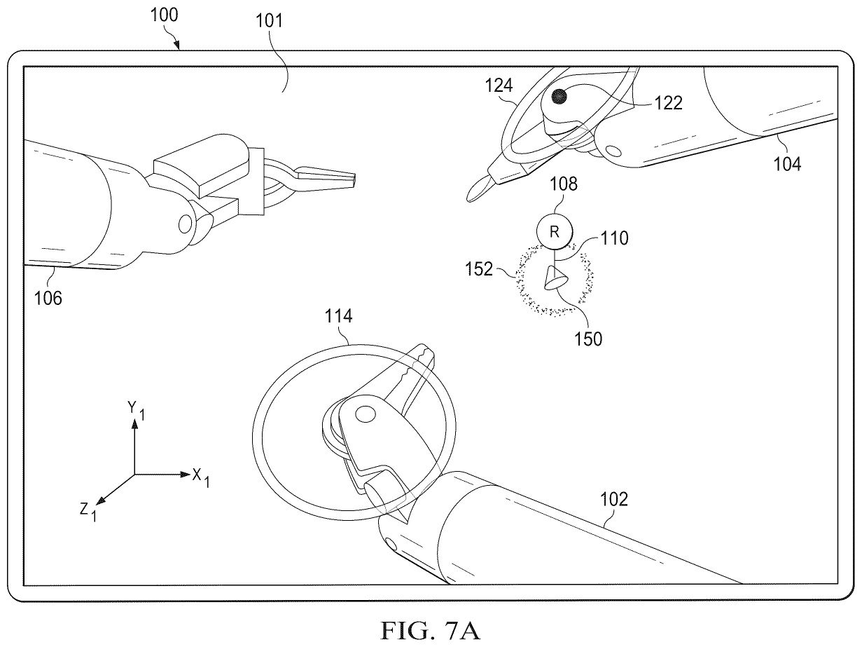

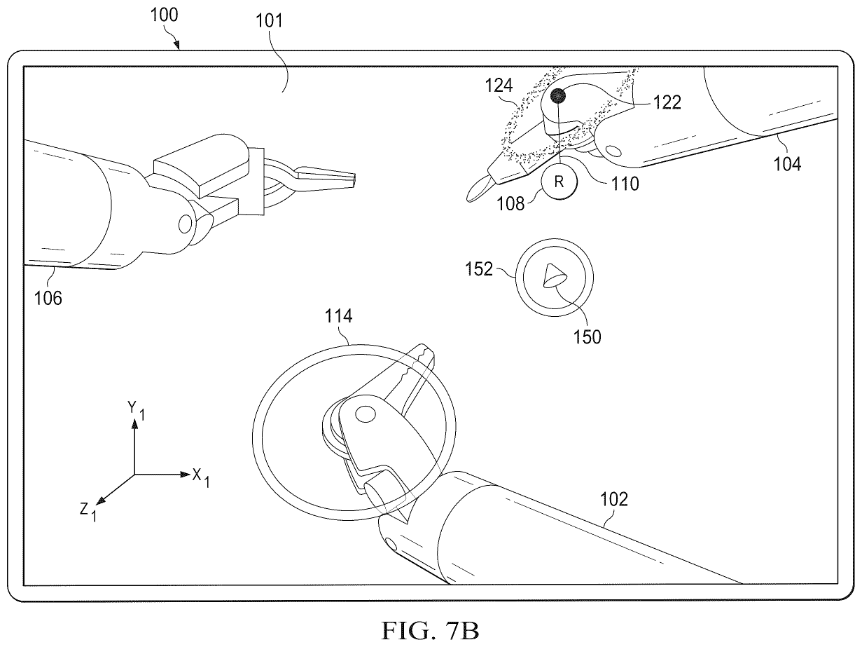

As shown in FIGS. 7A, the control device may, initially or at various times during the medical procedure, be unassigned from a tool. When the control device is unassigned, an unassigned control indicator 150 is displayed at the location of the control device 10 registered in the image frame of reference. The cursor 108 is tethered by the indicator line 110 to a keypoint of the indicator 150. The cursor 108 may include and “R” or “L” to indicate whether the cursor represents the right or left control device, respectively. A selection hoop 152 surrounding the unassigned control indicator 150 may be opaque and/or colored to indicate the association with the control device 10. At FIG. 7B, while in swap mode, the cursor 108 may be moved toward the keypoint 122 of tool 104. When the cursor enters the selection region 120 (suppressed in FIGS. 7A & 7B), the indicator line 110 snaps to the keypoint 122. As the cursor 108 is moved toward the keypoint 122, the appearance of the selection hoop may become altered, for example changing from translucent to opaque and/or changing from white to the selection color. The selection may be completed when the cursor 108 reaches the keypoint 122 or within a threshold distance from the keypoint 122.

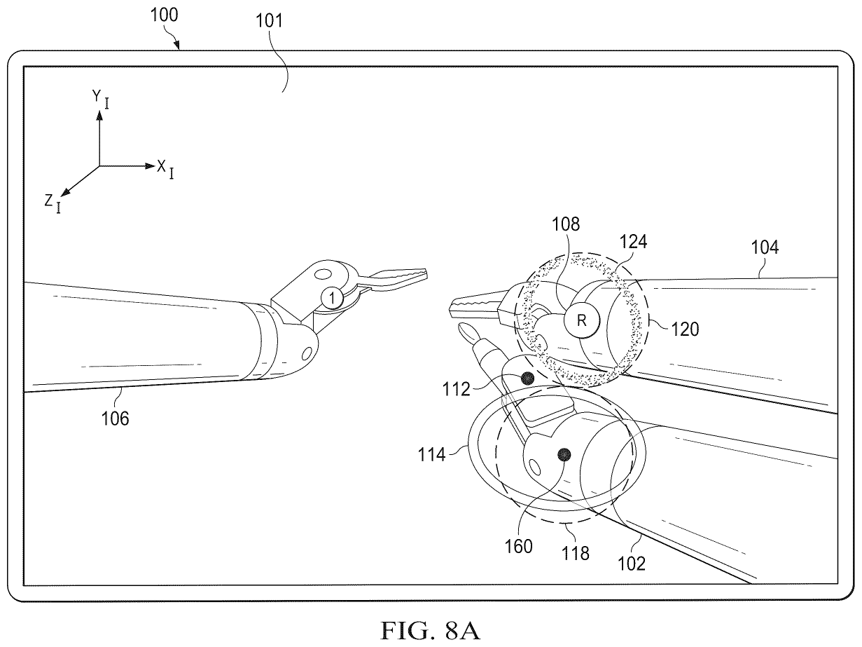

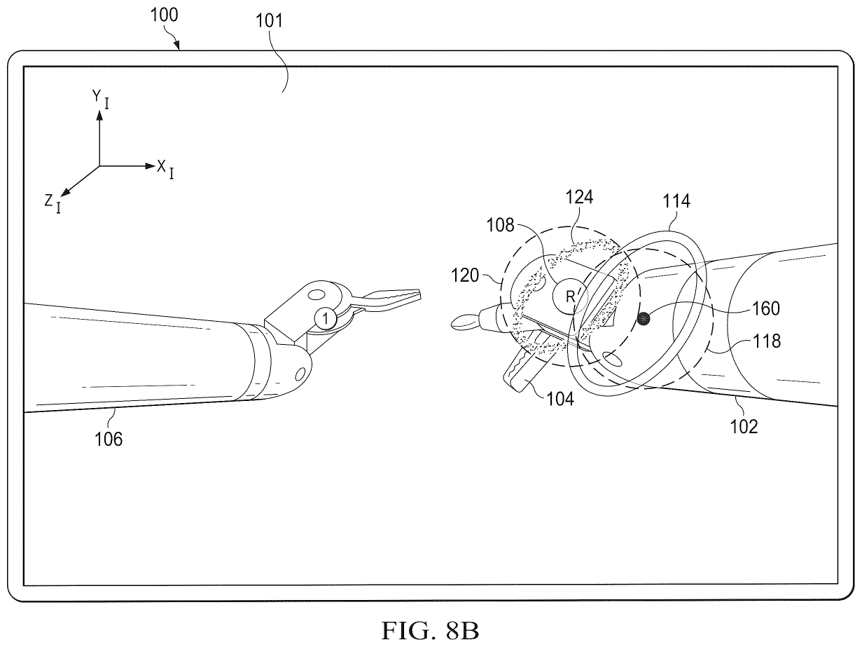

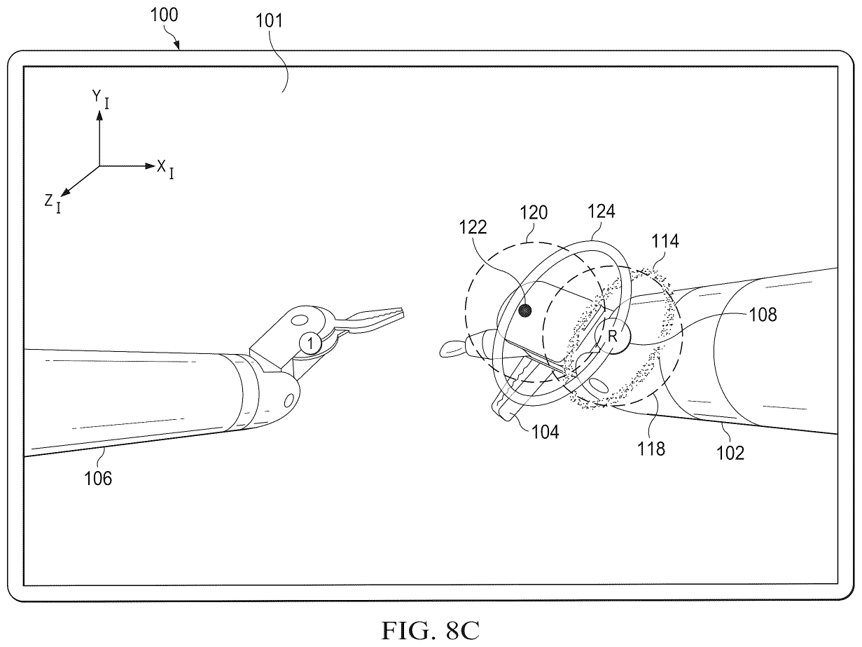

As shown in FIGS. 8A-8C, when tools are close together or overlap in the image 101 of the field of view, an alternative keypoint location may be provided for at least one of the tools. The selection region and the selection hoop for may be relocated to the alternative keypoint. For example, in FIG. 8A, the original keypoint 112 for tool 102 is sufficiently close to the keypoint 122 (obscured by cursor 108) for tool 104 in the image of the field of view that the selection regions surrounding the keypoints 112, 122 would overlap. The default locations for the keypoints may be at a distal clevis point or at another distal landmark of the tool. To prevent overlapping selection regions, an alternative keypoint 160, more proximal on the shaft of tool 102 and farther away from the keypoint 122, may reestablish the center of the selection region 118 and the selection hoop 114. At the alternative keypoint 160, the selection regions 118 and 120 may have minimal or no overlap. The increased spacing between the keypoints 122, 160 may provide the operator with greater clarity when moving the cursor 108 from keypoint 122 to keypoint 160.

In FIG. 8B, tool 102 almost entirely obstructs the view of tool 104 in the image 101 of the field of view. The cursor 108 is positioned at the keypoint 122 of the tool 104 and the approximate center of the selection region 120. In this example, the default keypoints 112, 122 and their respective selection regions may also be overlapping or substantially overlapping. Shifting the selection region 118 to the alternative keypoint 160 for tool 102 provides more space between the keypoints 122, 160 and provides the operator with greater clarity when selecting the target keypoint 160 and the target selection region 118 to reassign the control device 10 from tool 104 to tool 102. FIG. 8C illustrates the cursor 108 moved from the keypoint 122 on tool 104 to the keypoint 160 on the tool 102. The selection hoop 118 is opaque and rendered in the selection color indicating control has been switched and the tool 102 is now under the control of the control device 10. In some examples, where the tool and selection region overlap exceed a threshold overlap, a movement in a third lateral direction (e.g., Z-direction) may be used to select the tool closer or farther away in the image 101 of the field of view. In some examples, graphical depth cues such as sizing, shading, shadows, or occlusions may be applied to provide the sense of motion in the third lateral direction. Stereopsis is a primary depth cue for a stereoscopic display. Perspective foreshortening and depth aware blending of the hoops and cursor also help to clarify the depth presentation. In some examples, swapping to a new tool may be contingent on the cursor moving beyond a threshold distance away from the currently selected tool's selection region before an overlapping selection region for another tool may be selected. This may help avoid unintentional selection changes.

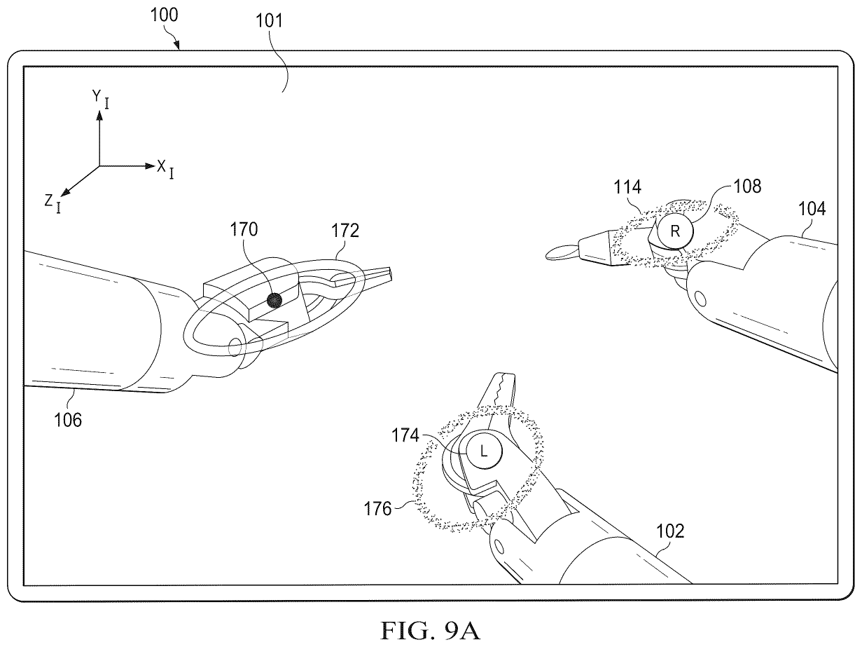

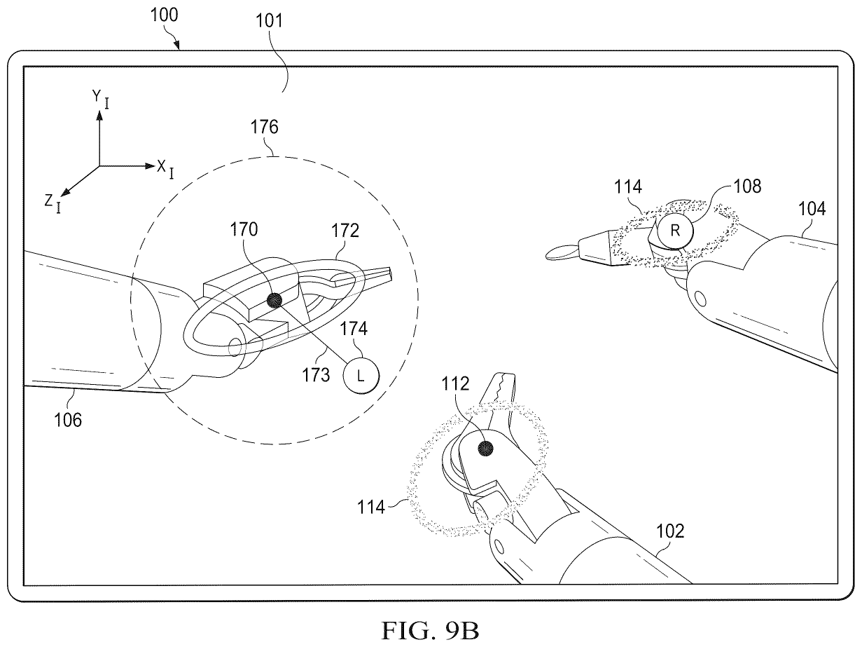

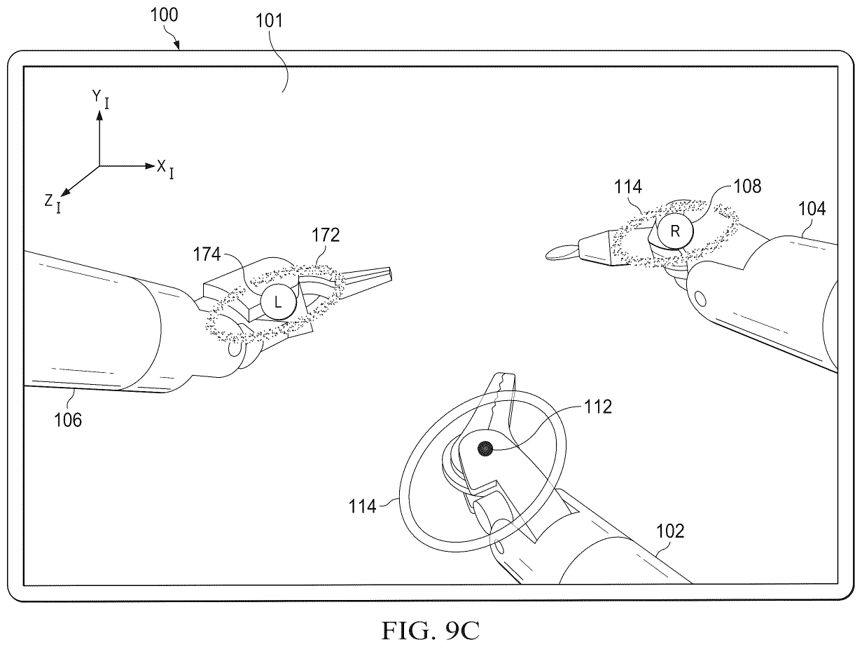

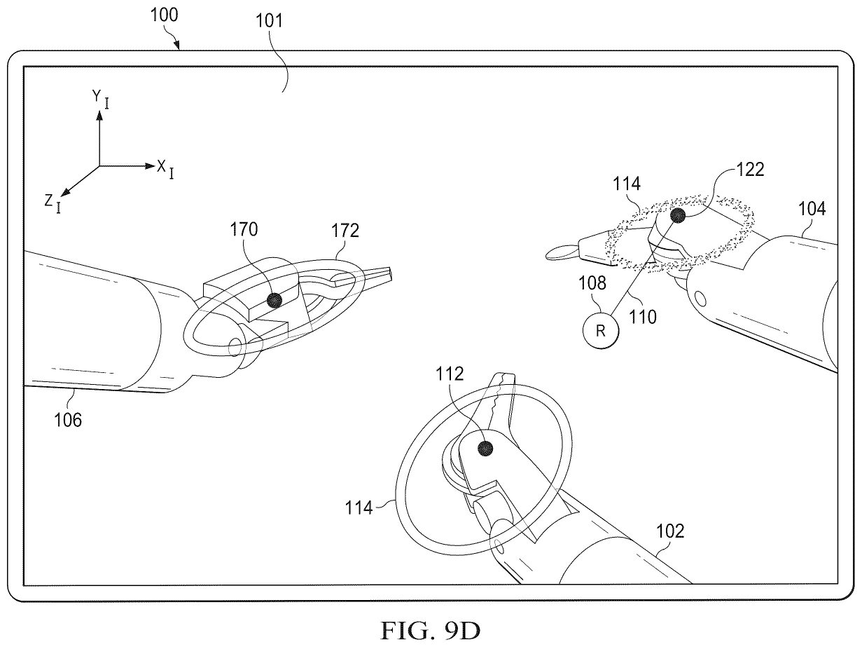

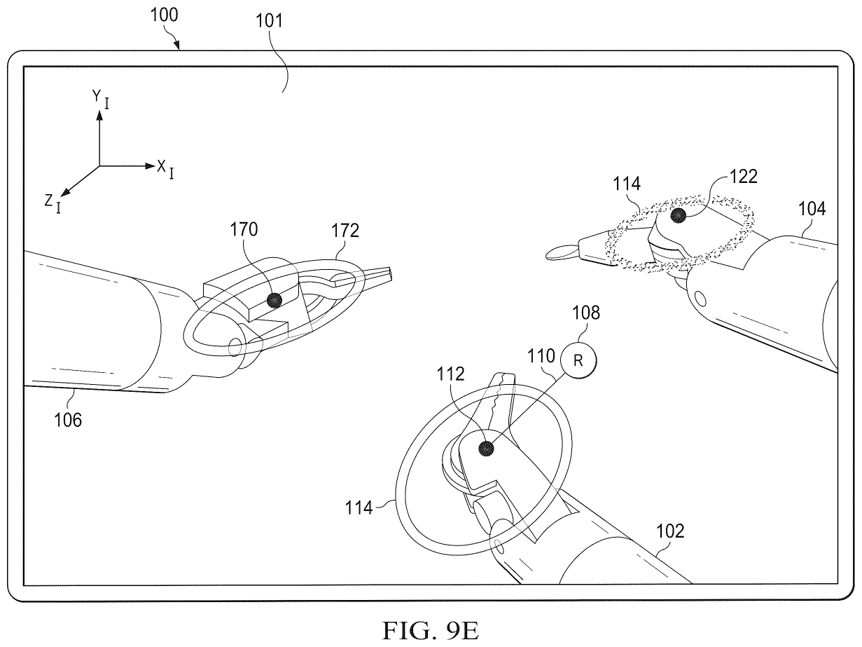

FIGS. 9A-9E illustrate the graphical user interface 100 as both left-and right-hand control devices switch control to different tools. At FIG. 9A, the right-hand control device 10 may initially be assigned to control the tool 104 and the left-hand control device 12 may initially be assigned to control the tool 102. Thus, the right cursor 108 is positioned at the keypoint 122 of the tool 104 and a left cursor 174 is positioned at the keypoint 112 of the tool 102. In the swap mode, as shown in FIG. 9B, the cursor 174 may be moved in the image frame of reference by moving the left-hand control device 12 in the operator frame of reference. As the cursor 174 moves toward a keypoint 170 on the tool 106 and enters a selection region 176 for the tool 106, an indictor line 173 may connect the cursor 174 to the keypoint 170. A gravity well associated with the selection region 176 may provide a haptic force to urge the control device 12 to move the cursor 174 toward the keypoint 170. As shown in FIG. 9C, with the cursor 174 at the keypoint 170 of the tool 106, a selection hoop 172 for the tool 106 is changed from translucent to opaque and/or is rendered in a selection color, thus indicating that the control device 12 has relinquished control of tool 102 and assumed control of tool 106. As shown in FIG. 9D, the cursor 108 may be moved in the image frame of reference by moving the right-hand control device 10 in the operator frame of reference. As the cursor 108 moves away from the keypoint 122 of the tool 104, the indicator line 110 appears between the cursor 108 and the keypoint 122 while the cursor is in the selection region 120. As shown in FIG. 9E, as the cursor 108 moves toward the keypoint 112 on the tool 104 and enters the selection region 118 for the tool 102, the indictor line 110 may connect the cursor 108 to the keypoint 112. The gravity well associated with the selection region 118 may provide a haptic force to urge the control device 10 to move the cursor 108 toward the keypoint 112. When the cursor 108 reaches the keypoint 112 of the tool 102, the selection hoop 114 for the tool 102 may be changed from translucent to opaque and/or be rendered in a selection color, thus indicating that the control device 10 has relinquished control of tool 104 and assumed control of tool 102.

In some examples, swapping may be prioritized to tools that are on manipulator arms that are on the same side of the endoscope as the operator's hand. In other words, a swapping preference may be to tools that are in the same direction relative to the endoscope or to tools that may be expected for a given hand. A swap may be permitted to tools on an opposite side of the endoscope, but, in that case, a close match between the pointing orientation of the control device and the pointing orientation of the selected tool may be required. In some examples, instrument control may be swapped to a control device of a second user console operated, for example, by a second operator.

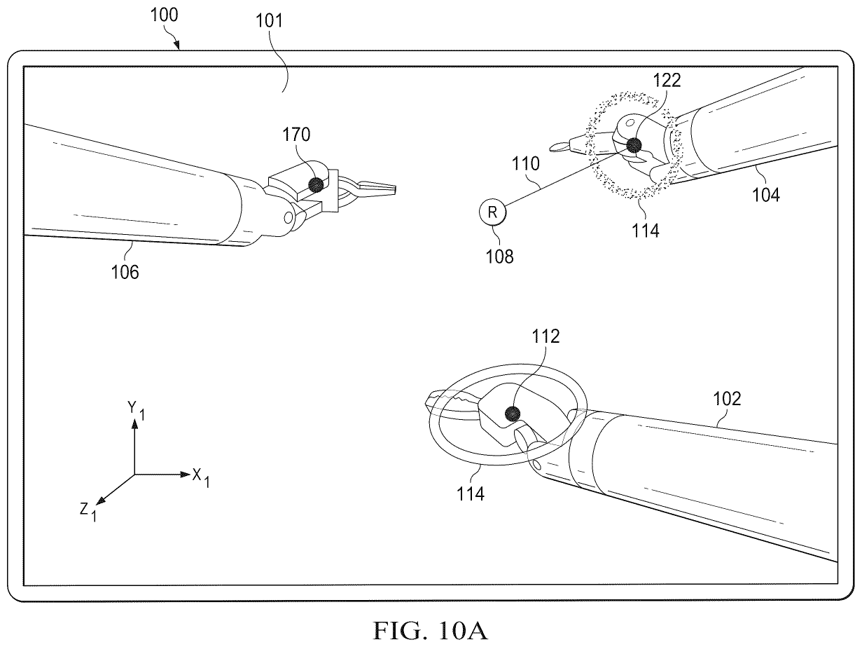

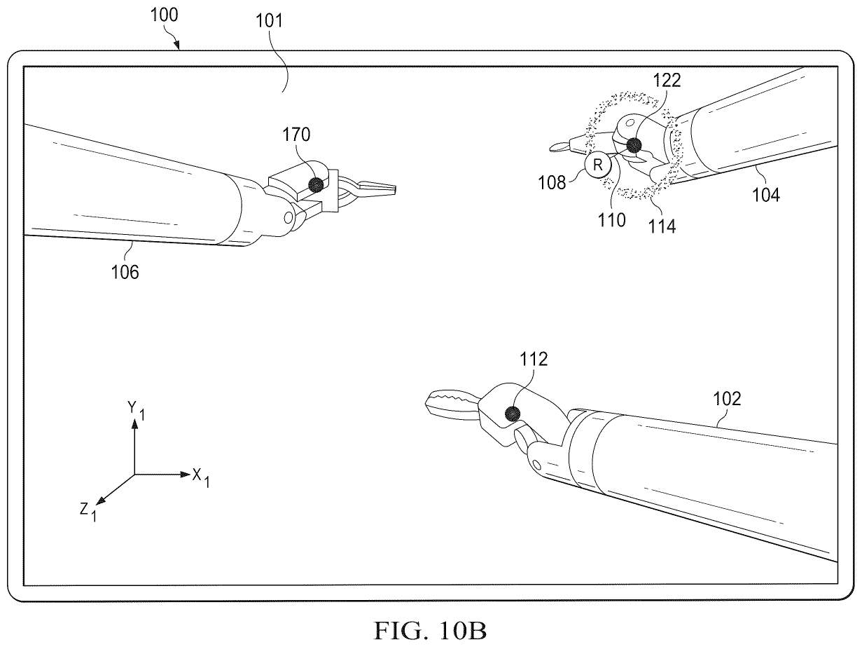

FIGS. 10A and 10B illustrate the graphical user interface 100 as a right-hand control device swap is cancelled. As shown in FIG. 10A, the right-hand control device 10 may initially be assigned to control the tool 104. In the swap mode, the cursor 108 may be moved in the image frame of reference by moving the left-hand control device 10 in the operator frame of reference. As the cursor 108 moves away from the keypoint 122 and toward the keypoint 170 on the tool 106 or the keypoint 112 on the tool 102, the indictor line 110 may connect the cursor 108 to the keypoint 122. If the swap is interrupted or cancelled, the cursor 108 may snap or quickly move back toward the keypoint 122, with the indicator line 110 contracting as the cursor 108 is returned to the keypoint. Interruptions may be caused by an operator's deliberate cancellation of the swap, movement of the endoscope, or movement and/or removal of a tool in the surgical environment. Cancellation may also be caused by actions indicating a user intent to cancel such as the cursor dwelling in the selection region, away from the keypoint, for longer than a threshold period of time or movement of the cursor out of the selection region and then returning the cursor to the selection region. Cancellation may also be caused by any of a variety of system events that may be incompatible with deliberate user intention to select a new tool, such as detection that the operator's head has lost contact with the operator console (e.g. “loss of head presence”), detection that the operator's hand is no longer engaged with the control device (e.g., “loss of hand presence”), interruption or loss of the endoscopic video signal, or loss of illumination in the field of view.

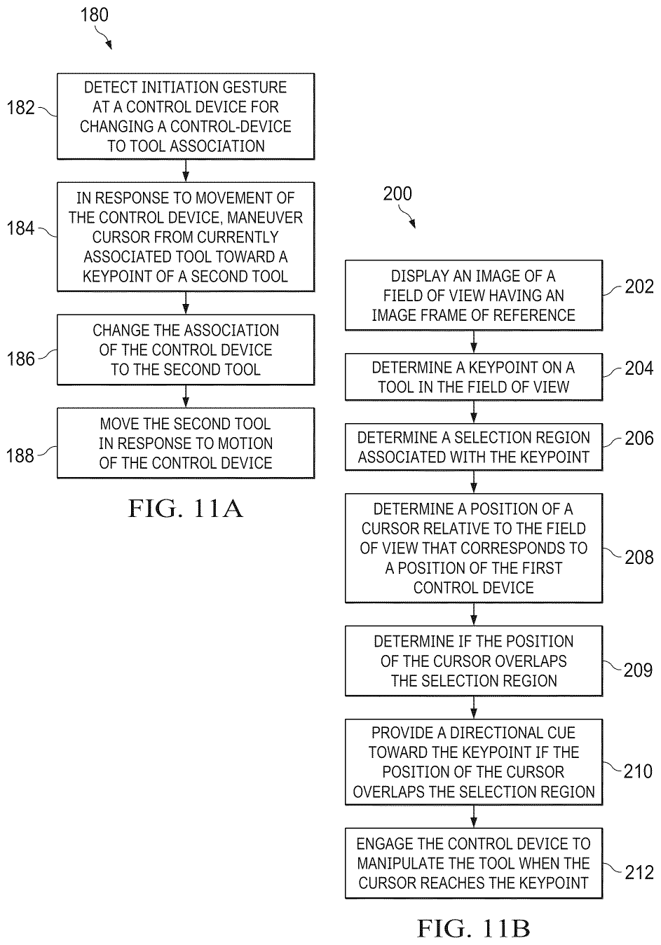

FIGS. 11A and 11B are flowcharts illustrating methods for taking control of a tool with a control device. The methods described herein are illustrated as a set of operations or processes and are described with continuing reference to the additional figures. Not all of the illustrated processes may be performed in all embodiments of the methods. Additionally, one or more processes that are not expressly illustrated in may be included before, after, in between, or as part of the illustrated processes. In some embodiments, one or more of the processes may be implemented, at least in part, in the form of executable code stored on non-transitory, tangible, machine-readable media that when run by one or more processors (e.g., the processors of a control system) may cause the one or more processors to perform one or more of the processes. In one or more embodiments, the processes may be performed by a control system.

In describing the method, reference may be made to FIGS. 2A-2C, but the same or similar processes may be used for assuming tool control in any other the examples described herein. The processes described may be performed during a swap mode of robot-assisted medical system in which movement of a control device may be used to change the tool controlled by the control device and movement of the control device does not cause a corresponding motion of a tool. If the medical system is in an instrument following mode in which the control device has active control of the motion of a tool, the system may exit the instrument follow mode and enter the swap mode to allow reassignment of a control device for controlling motion of a different tool.

With reference to FIG. 11A, at a process 182, an initiation gesture may be detected at a control device for changing a tool association for the control device. Intentional entry into the swap mode may be confirmed by the operator's performance of a confirmation gesture or generation of an initiation signal or command. For example, a confirmation gesture may be a double pull on a finger switch of the control device with the control device remaining otherwise stationary between the first and second pulls. The gesture may be right or left handed so that each hand can independently activate the swap feature. Other confirmation gestures may include depressing a foot pedal. In some examples a confirmation gesture performed on the hand control device may free the foot pedals to be used for other commands.

At a process 184, a cursor may be maneuvered, in response to movement of the control device, from the currently associated first tool toward a keypoint of a second tool. At a process 186, the association of the control device from the first tool to the second tool may be changed. This process is further described in FIG. 11B. At a process 188, after the association has been changed, the second tool may be moved in response to motion of the control device.

With reference to FIG. 11B, in some examples, the process of changing the association of a control device from one tool to another tool (e.g., process 186) may be include one or more of the processes described in method 200. At a process 202, an image of the field of view may be displayed. For example, an image 101 may be displayed on a display system 14. At a process 204, a keypoint on a tool in the field of view is determined. For example, the keypoint 122 may be recognized for the tool 104. At a process 206, a selection region associated with the keypoint is determined. The selection region may include the keypoint and may have an irregular or regular shaped boundary. For example, the selection region 120 may be associated with the keypoint 122. This process may evaluate a plurality of candidate keypoints in order to optimize the selection region size and compare alternative keypoints to determine an optimal configuration of keypoint locations and selection regions with minimal overlap. At a process 208, a position of a cursor relative to the field of view is determined. The two-dimensional position may correspond to a position of a control device (e.g., control device 10) in an operator frame of reference. For example, the cursor 108 is located at a two-dimensional position in the image frame of reference that corresponds to the position of the control device 10 in the registered operator frame of reference. The selection region 120 may also be determined in the perspective-projected frame of reference or coordinate space. The perspective-projected coordinate space may be referred to as normalized device coordinates (NDC). When a view volume is mapped to NDC coordinates, points within the view volume may be mapped to a cube volume with dimensions of [−1, 1] in X,Y,Z. Points that were formerly aligned along the perspective lines of the view volume may now be aligned in the XY plane, such that view apparent overlap can be evaluated in the XY plane without needing to reference the Z coordinate.

At a process 209, a determination may be made as to whether the position of the cursor overlaps the selection region. The cursor may overlap the selection region if it is located within or partially within the selection region. At a process 210, a directional cue may be provided to direct the cursor toward the keypoint if the cursor overlaps the selection region. For example, as the cursor 108 enters the selection region 120, a directional cue in the form of the graphic indicator line 110 may visually connect the cursor 108 to the keypoint 122, and a directional cue in the form of a haptic force may attract the control device 10 toward a position in the operator frame of reference that corresponds to the position of the keypoint 122 in the image frame of reference. The indicator line 110 may shorten as the cursor 108 and control device 10 moves, creating the visual impression that the indicator line 110 is pulling the cursor 108 toward the keypoint 122. At a process, 212, the control device may be engaged to manipulate the tool when the cursor reaches the keypoint or a threshold distance from the keypoint. The criteria for settling the engagement may include position and/or orientation criteria for matching of the control device with the controlled instruments. The criteria for settling the engagement may also include velocity criteria that indicate a slowing of the control device motion as the cursor nears the keypoint. For example, the control device 10 may be engaged to manipulate the tool 104 when the cursor 108 reaches the keypoint 122. The cursor 108 alignment with the keypoint 122 may be a confirmation cue that the control device 10 has been moved to its target position and orientation to begin manipulating the second tool. In some examples, the haptic force may urge the control device 10 toward a position and an orientation that matches the position, orientation, and/or grip angle of the tool 104 or its end effector. After the control device 10 takes control of the tool 104 and a hand position and orientation are confirmed to match the position and orientation of the tool 104 within an angular tolerance, a confirmation gesture may be performed prior to exiting the swap mode. In some examples, confirmation may include determining an axial direction of the tool relative to an imaging position of the endoscope and may include determining if the orientation of the control device matches an orientation of an end effector of the tool. In some examples, after the cursor 108 has settled on the keypoint 122, a squeeze on a gripping member of the control device may provide a grip indication, confirming the grip for the tool has been matched. After the confirmation gesture is received, the swap mode may be exited with or without a further exit gesture. After exiting the swap mode, the medical system may enter the instrument following mode of operation in which movement of the control device may cause movement of the tool 104.

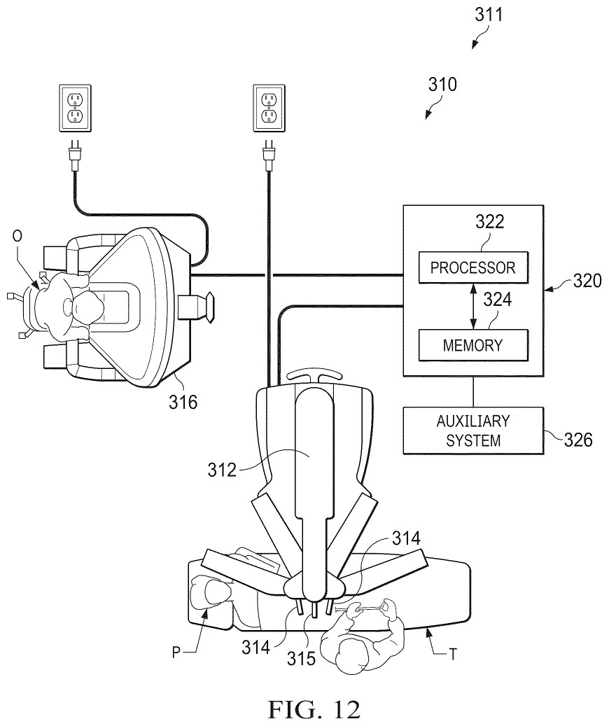

FIGS. 12-15 together provide an overview of a medical system 310 that may be used in, for example, medical procedures including diagnostic, therapeutic, or surgical procedures. The control swapping examples provided above may be used in the context of the medical system 310. The medical system 310 is located in a medical environment 311. The medical environment 311 is depicted as an operating room in FIG. 12. In other embodiments, the medical environment 311 may be an emergency room, a medical training environment, a medical laboratory, or some other type of environment in which any number of medical procedures or medical training procedures may take place. In still other embodiments, the medical environment 311 may include an operating room and a control area located outside of the operating room.

In one or more embodiments, the medical system 310 may be a robot-assisted medical system that is under the teleoperational control of an operator (e.g., a surgeon, a clinician, a physician, etc.). In alternative embodiments, the medical system 310 may be under the partial control of a computer programmed to perform the medical procedure or sub-procedure. In still other alternative embodiments, the medical system 310 may be a fully automated medical system that is under the full control of a computer programmed to perform the medical procedure or sub-procedure with the medical system 310. One example of the medical system 310 that may be used to implement the systems and techniques described in this disclosure is the da Vinci® Surgical System manufactured by Intuitive Surgical, Inc. of Sunnyvale, California.

As shown in FIG. 12, the medical system 310 generally includes an assembly 312, which may be mounted to or positioned near an operating table T on which a patient P is positioned. The assembly 312 may be referred to as a patient side cart, a surgical cart, or a surgical robot. In one or more embodiments, the assembly 312 may be a teleoperational assembly. The teleoperational assembly may be referred to as, for example, a teleoperational arm cart. A medical instrument system 314 and an endoscopic imaging system 315 are operably coupled to the assembly 312. An operator input system 316 allows an operator O or other type of clinician to view images of or representing the surgical site and to control the operation of the medical instrument system 314 and/or the endoscopic imaging system 315.

The medical instrument system 314 may comprise one or more medical instruments. In embodiments in which the medical instrument system 314 comprises a plurality of medical instruments, the plurality of medical instruments may include multiple of the same medical instrument and/or multiple different medical instruments. Similarly, the endoscopic imaging system 315 may comprise one or more endoscopes. In the case of a plurality of endoscopes, the plurality of endoscopes may include multiple of the same endoscope and/or multiple different endoscopes.

The operator input system 316 may be located at a operator's control console, which may be located in the same room as operating table T. In some embodiments, the operator O and the operator input system 316 may be located in a different room or a completely different building from the patient P. The operator input system 316 generally includes one or more control device(s) for controlling the medical instrument system 314. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and other types of input devices.

In some embodiments, the control device(s) will be provided with the same degrees of freedom as the medical instrument(s) of the medical instrument system 314 to provide the operator with telepresence, which is the perception that the control device(s) are integral with the instruments so that the operator has a strong sense of directly controlling instruments as if present at the surgical site. In other embodiments, the control device(s) may have more or fewer degrees of freedom than the associated medical instruments and still provide the operator with telepresence. In some embodiments, the control device(s) are manual input devices that are movable with six degrees of freedom, and which may also include an actuatable handle for actuating instruments (for example, for closing grasping jaw end effectors, applying an electrical potential to an electrode, delivering a medicinal treatment, and actuating other types of instruments).

The assembly 312 supports and manipulates the medical instrument system 314 while the operator O views the surgical site through the operator input system 316. An image of the surgical site may be obtained by the endoscopic imaging system 315, which may be manipulated by the assembly 312. The assembly 312 may comprise endoscopic imaging systems 315 and may similarly comprise multiple medical instrument systems 314 as well. The number of medical instrument systems 314 used at one time will generally depend on the diagnostic or surgical procedure to be performed and on space constraints within the operating room, among other factors. The assembly 312 may include a kinematic structure of one or more non-servo controlled links (e.g., one or more links that may be manually positioned and locked in place, generally referred to as a set-up structure) and a manipulator. When the manipulator takes the form of a teleoperational manipulator, the assembly 312 is a teleoperational assembly. The assembly 312 includes a plurality of motors that drive inputs on the medical instrument system 314. In an embodiment, these motors move in response to commands from a control system (e.g., control system 320). The motors include drive systems which when coupled to the medical instrument system 314 may advance a medical instrument into a naturally or surgically created anatomical orifice. Other motorized drive systems may move the distal end of said medical instrument in multiple degrees of freedom, which may include three degrees of linear motion (e.g., linear motion along the X, Y, Z Cartesian axes) and three degrees of rotational motion (e.g., rotation about the X, Y, Z Cartesian axes). Additionally, the motors may be used to actuate an articulable end effector of the medical instrument for grasping tissue in the jaws of a biopsy device or the like. Medical instruments of the medical instrument system 314 may include end effectors having a single working member such as a scalpel, a blunt blade, an optical fiber, or an electrode. Other end effectors may include, for example, forceps, graspers, scissors, or clip appliers.

The medical system 310 also includes a control system 320. The control system 320 includes at least one memory 324 and at least one processor 322 for effecting control between the medical instrument system 314, the operator input system 316, and other auxiliary systems 326 which may include, for example, imaging systems, audio systems, fluid delivery systems, display systems, illumination systems, steering control systems, irrigation systems, and/or suction systems. A clinician may circulate within the medical environment 11 and may access, for example, the assembly 312 during a set up procedure or view a display of the auxiliary system 326 from the patient bedside.

Though depicted as being external to the assembly 312 in FIG. 12, the control system 320 may, in some embodiments, be contained wholly within the assembly 312. The control system 320 also includes programmed instructions (e.g., stored on a non-transitory, computer-readable medium) to implement some or all of the methods described in accordance with aspects disclosed herein. While the control system 320 is shown as a single block in the simplified schematic of FIG. 12, the control system 320 may include two or more data processing circuits with one portion of the processing optionally being performed on or adjacent the assembly 312, another portion of the processing being performed at the operator input system 316, and the like.

Any of a wide variety of centralized or distributed data processing architectures may be employed. Similarly, the programmed instructions may be implemented as a number of separate programs or subroutines, or they may be integrated into a number of other aspects of the systems described herein, including teleoperational systems. In one embodiment, the control system 320 supports wireless communication protocols such as Bluetooth, IrDA, HomeRF, IEEE 802.11, DECT, and Wireless Telemetry.

In some embodiments, control system 320 may include one or more servo controllers that receive force and/or torque feedback from the medical instrument system 314. Responsive to the feedback, the servo controllers transmit signals to the operator input system 316. The servo controller(s) may also transmit signals instructing assembly 312 to move the medical instrument system(s) 314 and/or endoscopic imaging system 315 which extend into an internal surgical site within the patient body via openings in the body. Any suitable conventional or specialized servo controller may be used. A servo controller may be separate from, or integrated with, assembly 312. In some embodiments, the servo controller and assembly 312 are provided as part of a teleoperational arm cart positioned adjacent to the patient's body.

The control system 320 can be coupled with the endoscopic imaging system 315 and can include a processor to process captured images for subsequent display, such as to an operator on the operator's control console, or on another suitable display located locally and/or remotely. For example, where a stereoscopic endoscope is used, the control system 320 can process the captured images to present the operator with coordinated stereo images of the surgical site. Such coordination can include alignment between the opposing images and can include adjusting the stereo working distance of the stereoscopic endoscope.

In alternative embodiments, the medical system 310 may include more than one assembly 312 and/or more than one operator input system 316. The exact number of assemblies 312 will depend on the surgical procedure and the space constraints within the operating room, among other factors. The operator input systems 316 may be collocated or they may be positioned in separate locations. Multiple operator input systems 316 allow more than one operator to control one or more assemblies 312 in various combinations. The medical system 310 may also be used to train and rehearse medical procedures.

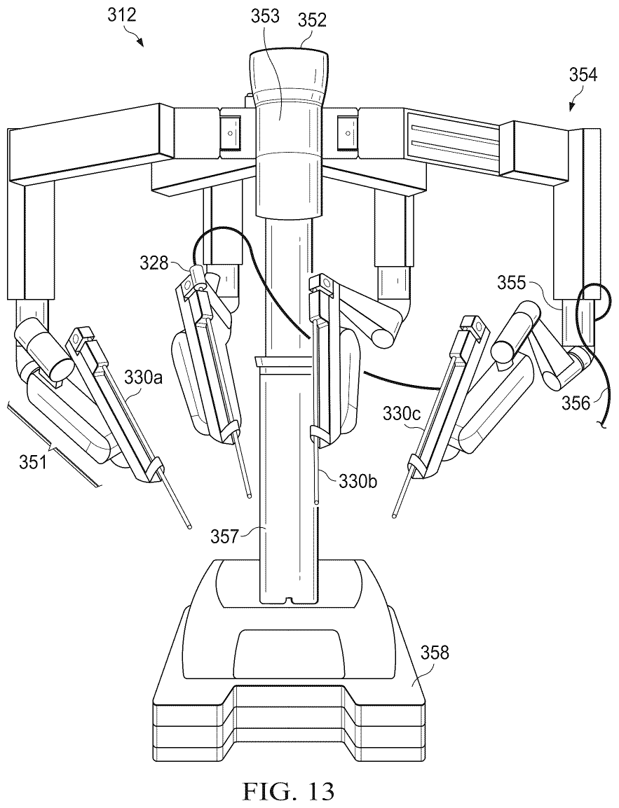

FIG. 13 is a perspective view of one embodiment of an assembly 312 which may be referred to as a patient side cart, surgical cart, teleoperational arm cart, manipulator assembly or surgical robot. The assembly 312 shown provides for the manipulation of three surgical tools 330a, 330b, and 330c (e.g., medical instrument systems 314) and an imaging device 328 (e.g., endoscopic imaging system 315), such as a stereoscopic endoscope used for the capture of images of the site of the procedure. The imaging device may transmit signals over a cable 356 to the control system 320. Manipulation is provided by teleoperative mechanisms having a number of joints. The imaging device 328 and the surgical tools 330a-c can be positioned and manipulated through incisions in the patient so that a kinematic remote center is maintained at the incision to minimize the size of the incision. Images of the surgical site can include images of the distal ends of the surgical tools 330a-c when they are positioned within the field of view of the imaging device 328.

The assembly 312 includes a drivable base 358. The drivable base 358 is connected to a telescoping column 357, which allows for adjustment of the height of arms 354. The arms 354 may include a rotating joint 355 that both rotates and moves up and down. Each of the arms 354 may be connected to an orienting platform 353. The arms 354 may be labeled to facilitate trouble shooting. For example, each of the arms 354 may be emblazoned with a different number, letter, symbol, other identifier, or combinations thereof. The orienting platform 353 may be capable of 360 degrees of rotation. The assembly 312 may also include a telescoping horizontal cantilever 352 for moving the orienting platform 353 in a horizontal direction.

In the present example, each of the arms 354 connects to a manipulator arm 351. The manipulator arms 351 may connect directly to a medical instrument, e.g., one of the surgical tools 330a-c. The manipulator arms 351 may be teleoperable. In some examples, the arms 354 connecting to the orienting platform 353 may not be teleoperable. Rather, such arms 354 may be positioned as desired before the operator O begins operation with the teleoperative components. Throughout a surgical procedure, medical instruments may be removed and replaced with other instruments such that instrument to arm associations may change during the procedure.

Endoscopic imaging systems (e.g., endoscopic imaging system 315 and imaging device 328) may be provided in a variety of configurations including rigid or flexible endoscopes. Rigid endoscopes include a rigid tube housing a relay lens system for transmitting an image from a distal end to a proximal end of the endoscope. Flexible endoscopes transmit images using one or more flexible optical fibers. Digital image-based endoscopes have a “chip on the tip” design in which a distal digital sensor such as a one or more charge-coupled device (CCD) or a complementary metal oxide semiconductor (CMOS) device store image data. Endoscopic imaging systems may provide two-or three-dimensional images to the viewer. Two-dimensional images may provide limited depth perception. Three-dimensional stereo endoscopic images may provide the viewer with more accurate depth perception. Stereo endoscopic instruments employ stereo cameras to capture stereo images of the patient anatomy. An endoscopic instrument may be a fully sterilizable assembly with the endoscope cable, handle and shaft all rigidly coupled and hermetically sealed.

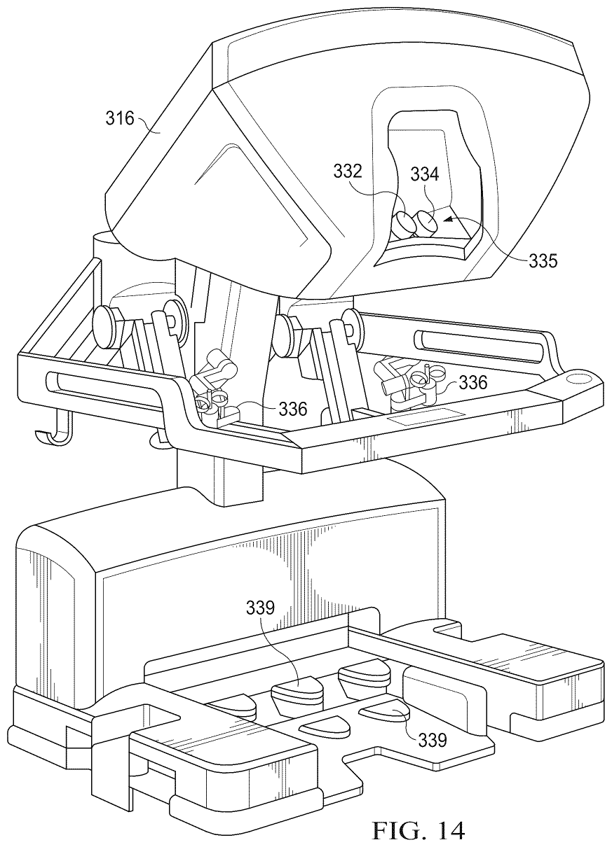

FIG. 14 is a perspective view of an embodiment of the operator input system 316 at the operator's control console. The operator input system 316 includes a display system with a left eye display 332 and a right eye display 334 for presenting the operator O with a coordinated stereo view of the surgical environment that enables depth perception. The left and right eye displays 332, 332 may be components of a display system 335 (e.g., the display system 14). In other embodiments, the display system 335 may include one or more other types of displays. The display system 335 may present images captured, for example, by the imaging system 315 to display the endoscopic field of view to the operator. The endoscopic field of view may be augmented by virtual or synthetic menus, indicators, and/or other graphical or textual information to provide additional information to the viewer.

The operator input system 316 further includes one or more input control devices 336, which in turn cause the assembly 312 to manipulate one or more instruments of the endoscopic imaging system 315 and/or medical instrument system 314. The input control devices 336 can provide the same degrees of freedom as their associated instruments to provide the operator O with telepresence, or the perception that the input control devices 336 are integral with said instruments so that the operator has a strong sense of directly controlling the instruments. To this end, position, force, and tactile feedback sensors (not shown) may be employed to transmit position, force, and tactile sensations from the medical instruments, e.g., surgical tools 330a-c, or imaging device 328, back to the operator's hands through the input control devices 336. Input control devices 339 are foot pedals that receive input from a user's foot. Aspects of the operator input system 316, the assembly 312, and the auxiliary systems 326 may be adjustable and customizable to meet the physical needs, skill level, or preferences of the operator O.

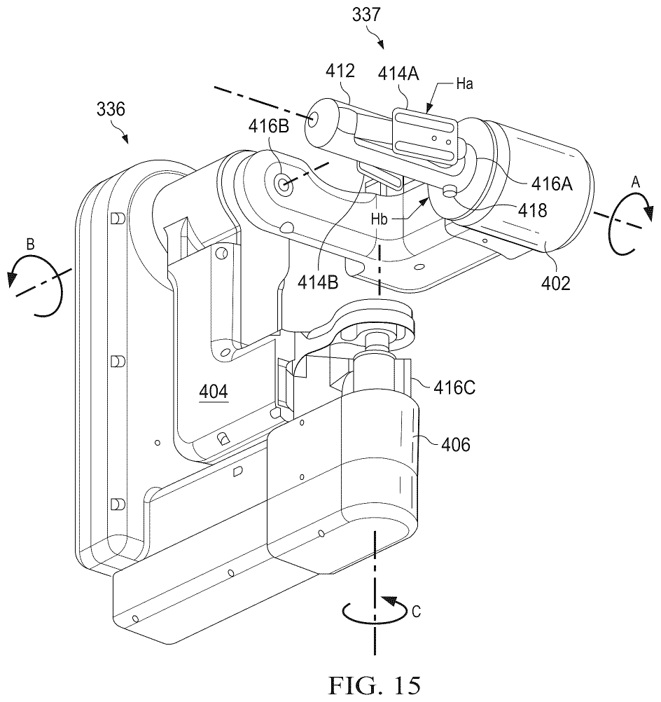

Referring now to 15, shown therein is a perspective view of an embodiment of one of the control devices 336 with a finger assembly 337. Embodiments of the control device 336 as shown in FIG. 15 may be used as the control devices 10, 12. The control device 336 is a gimbaled device that pivotally supports the assembly 337, which may include a touch sensitive handle to generate control signals that are used to control the assembly 302 and the robotic medical tools.

The depicted master control device 336 includes first, second, and third gimbal members 402, 404, and 406. The touch sensitive handle provided by finger assembly 304 includes a tubular support structure 412, a first grip 414A, and a second grip 414B. The first grip 414A and the second grip 414B are supported at one end by the structure 412. In some embodiments, the grips 414 may include loops of material that help secure the physician's fingers in place relative to the structure of the grips. Additionally, some embodiments may include more than two grips connected to the support structure 412 or two grips 414 and another control mechanism, like a button, switch, track pad, or scroll-wheel. For example, the control device 336 may include a button 418 that may be activated by the physician to switch control modes or perform a particular action. As shown, the button 418 is mounted at a proximal end of the support structure 412, disposed between the grips 414, such that it can be actuated when a hand grips the support structure 412. The button 418 may include a redundant button or a similar but non-redundant button disposed on the opposite side of the support structure 412. However, one or more similar buttons may be positioned elsewhere in other embodiments. The finger assembly 337 can be rotated about axis A, illustrated in FIG. 15. The grips 414A and 414B can be squeezed or pinched together about the tubular structure 412. The “pinching” or grasping degree of freedom in the grips is indicated by arrows Ha and Hb. These or other movements of the grips 414 relative to the support structure 412 may provide commands to manipulate the tools 330a-c.

The finger assembly 337 is rotatably supported by the first gimbal member 402 by means of a rotational joint 416A. The first gimbal member 402 is in turn, rotatably supported about axis B by the second gimbal member 404 by means of the rotational joint 416B. Similarly, the second gimbal member 404 is rotatably supported about axis C by the third gimbal member 406 using a rotational joint 416C. In this manner, the control device 336 allows the finger assembly 337 to be moved and oriented in the workspace using three degrees of freedom.

The movements in the gimbals of the master control device 336 to reorient the finger assembly 337 in space can be translated into control signals to control an arm/tool combination. For example, the rotational motion of the finger assembly 337 about axis A in FIG. 15 may be used to roll instrument 330a about its shaft axis. Alternatively or additionally, the squeezing motion of the grips 414A, 414B over their freedom of movement indicated by arrows Ha and Hb, may be used to command a grasping motion with forceps, or a cutting motion with scissors, or control the flow of fluids through the suction/irrigator robotic medical tool positioned at the interventional site, for example. The grips 414 may be passively biased to spring open, providing a restoring force to release forceps, open scissors, etc.

To sense the movements in the touch sensitive handle and generate controls signals for the instruments 330, sensors can be mounted in the finger assembly 337 as well as the gimbal members of the control device 336. Exemplary sensors may include a Hall effect transducer, a potentiometer, an encoder, or the like.

As described below in more detail, some procedures may require more instruments at the interventional site than the operator has hands or than the operator console 316 has control devices 336. Additionally, as in shown in FIG. 13, some embodiments of the assembly 312 may include five tools, while the console 316 only includes two control devices 336. Accordingly, when the operator wants to switch assignment of one of the master control devices, there are potentially three tools that the physician may want to control. Care should be taken to ensure that the correct instrument is selected from the three unassigned instruments when the physician wants to reassign control. As described herein, the console 316 includes features that permit the operator to change the control assignment between arms and/or tools and control devices, such that a particular control device can be reassigned from controlling a first tool to controlling a second tool, for example, as the operator deems necessary. In this way, a single operator may more effectively utilize more arms/instruments.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

Any alterations and further modifications to the described devices, systems, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately.

Various systems and portions of systems have been described in terms of their state in three-dimensional space. As used herein, the term “position” refers to the location of an object or a portion of an object in a three-dimensional space (e.g., three degrees of translational freedom along Cartesian X, Y, Z coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom).

Although some of the examples described herein refer to surgical procedures or instruments, or medical procedures and medical instruments, the techniques disclosed optionally apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system,” are analogous.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A medical system comprising:
a display system;
an input system including a first control device; and
a control system, wherein the control system includes a processing unit including one or more processors, and wherein the processing unit is configured to:
display, on the display system, an image of a field of view of a surgical environment, wherein the image is generated by an imaging component,
determine a first keypoint on a first tool in the surgical environment,
determine a selection region associated with the first keypoint of the first tool,
determine a position of a cursor relative to the field of view, the position of the cursor corresponding to a position of the first control device,
determine if the position of the cursor overlaps the selection region,
provide a directional cue if the position of the cursor overlaps the selection region to direct the cursor toward the first keypoint, and
engage the first control device to manipulate the first tool when the cursor reaches the first keypoint.

2. The medical system of claim 1, wherein providing the directional cue includes providing a haptic force to the first control device.

3. The medical system of claim 1, wherein providing the directional cue includes displaying a zipline graphic that extends between the cursor and the first keypoint.

4. The medical system of claim 1, wherein the cursor includes an identification symbol representing the first control device.

5. The medical system of claim 1, wherein the first control device is manipulatable in more than two translational degrees of freedom.

6. The medical system of claim 1, further comprising:
a second control device manipulatable in more than two translational degrees of freedom.

7. The medical system of claim 1, wherein the processing unit is further configured to:
receive an initiation signal to enter a swap mode;
responsive to movement of the first control device, move the cursor from the first keypoint toward a second tool in the image of the field of view; and
engage the first control device to manipulate the second tool when the cursor reaches a second keypoint of the second tool.

8. The medical system of claim 7, wherein a second selection region is associated with the second keypoint of the second tool and wherein the processing unit is further configured to provide a second directional cue when the position of the cursor overlaps the second selection region.

9. The medical system of claim 7, wherein the initiation signal is generated based on receipt of a user input at the first control device.

10. The medical system of claim 9, wherein the initiation signal is a double input at a finger switch of the first control device while the first control device is otherwise stationary.

11. The medical system of claim 7, wherein the processing unit is further configured to receive a grip indication before the first control device is engaged to manipulate the second tool.

12. The medical system of claim 7, wherein a dimension of the selection region is changeable based on a proximity of the first tool to the second tool.

13. The medical system of claim 7, wherein a location of the second keypoint of the second tool is adjustable based on a proximity of the first tool to the second tool.

14. The medical system of claim 7, wherein if the first keypoint is within a threshold distance from the second keypoint, evaluate a three-dimensional motion of the first control device and engage the first control device to manipulate the second tool based on the three-dimensional motion of the first control device.

15. The medical system of claim 1, wherein the first tool is outside the field of view and the selection region is at least partially within the field of view.

16. The medical system of claim 1, wherein the first tool is a medical instrument or is a menu displayed on the display system.

17. (canceled)

18. The medical system of claim 1, wherein the selection region is centered on the first keypoint.

19. The medical system of claim 1, wherein determining the position of the cursor includes determining an affine transformation of the first control device to affect motion scaling and a position offset of an initial cursor position.

20. The medical system of claim 1, wherein the processing unit is further configured to:
determine an axial direction of the first tool relative to an imaging position of the imaging component; and
engage the first control device to manipulate the first tool when an orientation of the first control device matches an orientation of an end effector of the first tool within an angular tolerance.

21. The medical system of claim 1,
wherein determining the position of the cursor relative to the field of view includes mapping the cursor to a normalized device coordinate space of a single eyepiece of a stereoscopic imaging system,
wherein determining a selection region includes mapping the selection region to the normalized device coordinate space of the single eyepiece of a stereoscopic imaging system, and
wherein determining if the position of the cursor overlaps the selection region is determined independent of depth.

22-46. (canceled)
